SEQUENCE LISTING (50 sequences)

SEQ ID NO: 1 - protein, 9 aa, Homo sapiens
HMTEVVRHC

SEQ ID NO: 2 - protein, 365 aa, Homo sapiens [sequence omitted]

SEQ ID NO: 3 - protein, 11 aa, Homo sapiens
CAVNQAGTALI

SEQ ID NO: 4 - protein, 11 aa, Homo sapiens
CASMGTGDEAF

SEQ ID NO: 5 - protein, 265 aa, synthetic construct
  REGION 1..265: note = Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVNQAGTALIFGKGTTLSVSSNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 6 - protein, 304 aa, synthetic construct
  REGION 1..304: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASMGTGDEAFFGQGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 7 - protein, 5 aa, Homo sapiens
DSVNN

SEQ ID NO: 8 - protein, 5 aa, Homo sapiens
IPSGT

SEQ ID NO: 9 - protein, 5 aa, Homo sapiens
SNHLY

SEQ ID NO: 10 - protein, 6 aa, Homo sapiens
FYNNEI

SEQ ID NO: 11 - protein, 129 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVNQAGTALIFGKGTTLSVSSN

SEQ ID NO: 12 - protein, 131 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASMGTGDEAFFGQGTRLTVV

SEQ ID NO: 13 - protein, 32 aa, synthetic construct
  REGION 1..32: note = Synthetic
RAKRSGSGAPVKQTLNFDLLKLAGDVESNPGP

SEQ ID NO: 14 - DNA, 22 bp, Homo sapiens
  source 1..22: mol_type = genomic DNA
ggcagacaggaccccttgctgg

SEQ ID NO: 15 - DNA, 24 bp, Homo sapiens
  source 1..24: mol_type = genomic DNA
cttttctcgaccagcttgacatca

SEQ ID NO: 16 - DNA, 1803 bp, synthetic construct
  misc_feature 1..1803: note = Synthetic
  source 1..1803: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcacgtgccaagcgatccggaagcggagcccctgtaaagcagactttgaattttgaccttctcaagttggcgggagacgtcgagtccaaccctgggcccatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaatatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 17 - DNA, 4301 bp, synthetic construct
  misc_feature 1..4301: note = Synthetic
  source 1..4301: mol_type = other DNA
ttagcgagatccggtggagccgggtccggcggtgccgtccacggcagaattggacgactgagcgcgggatctggcgaaggcgatgggggtcttgaaggcgtgctggtactccacgatgcccagctcggtgttgctgtgcagctcctccacgcggcggaaggcgaacatggggcccccgttctgcaggatgctggggtggatggcgctcttgaagtgcatgtggctgtccaccacgaagctgtagtagccgccgtcgcgcaggctgaaggtgcgggcgaagctgcccaccagcacgttatcgcccatggggtgcaggtgctccacggtggcgttgctgcggatgatcttgtcggtgaagatcacgctgtcctcggggaagccggtgcccaccaccttgaagtcgccgatcacgcggccggcctcgtagcggtagctgaagctcacgtgcagcacgccgccgtcctcgtacttctcgatgcgggtgttggtgtagccgccgttgttgatggcgtgcaggaaggggttctcgtagccgctggggtaggtgccgaagtggtagaagccgtagcccatcacgtggctcagcaggtaggggctgaaggtcagggcgcctttggtgctcttcatcttgttggtcatgcggccctgcttgggggtgccctctccgccgcccaccagctcgaactccacgccgttcagggtgccggtgatgcggcactcgatctccatggcgggcaggccgctctcgtcgctctccatggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgccgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcacgtgccaagcgatccggaagcggagcccctgtaaagcagactttgaattttgaccttctcaagttggcgggagacgtcgagtccaaccctgggcccatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaatatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagttga

SEQ ID NO: 18 - protein, 393 aa, Homo sapiens [sequence omitted]

SEQ ID NO: 19 - protein, 393 aa, Homo sapiens [sequence omitted]

SEQ ID NO: 20 - protein, 9 aa, Homo sapiens
HMTEVVRRC

SEQ ID NO: 21 - protein, 19 aa, Homo sapiens
MDTWLVCWAIFSLLKAGLT

SEQ ID NO: 22 - protein, 20 aa, Homo sapiens
MKRILGALLGLLSAQVCCVR

SEQ ID NO: 23 - protein, 112 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASMGTGDEAFFGQGTRLTVV

SEQ ID NO: 24 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVNQAGTALIFGKGTTLSVSSN

SEQ ID NO: 25 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 26 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 27 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 28 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 29 - DNA, 15 bp, Homo sapiens
  source 1..15: mol_type = genomic DNA
gactctgtgaacaat

SEQ ID NO: 30 - DNA, 15 bp, Homo sapiens
  source 1..15: mol_type = genomic DNA
attccctcagggaca

SEQ ID NO: 31 - DNA, 33 bp, Homo sapiens
  source 1..33: mol_type = genomic DNA
tgtgctgttaaccaggcaggaactgctctgatc

SEQ ID NO: 32 - DNA, 15 bp, Homo sapiens
  source 1..15: mol_type = genomic DNA
tctaatcacttatac

SEQ ID NO: 33 - DNA, 18 bp, Homo sapiens
  source 1..18: mol_type = genomic DNA
ttttataataatgaaatc

SEQ ID NO: 34 - DNA, 33 bp, Homo sapiens
  source 1..33: mol_type = genomic DNA
tgtgccagcatggggacaggagatgaagctttc

SEQ ID NO: 35 - DNA, 387 bp, Homo sapiens
  source 1..387: mol_type = genomic DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaat

SEQ ID NO: 36 - DNA, 393 bp, Homo sapiens
  source 1..393: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgta

SEQ ID NO: 37 - DNA, 807 bp, Homo sapiens
  source 1..807: mol_type = genomic DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 38 - DNA, 921 bp, Homo sapiens
  source 1..921: mol_type = genomic DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgtagacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 39 - DNA, 795 bp, synthetic construct
  misc_feature 1..795: note = Synthetic
  source 1..795: mol_type = other DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaatatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 40 - DNA, 912 bp, synthetic construct
  misc_feature 1..912: note = Synthetic
  source 1..912: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattca

SEQ ID NO: 41 - DNA, 327 bp, Homo sapiens
  source 1..327: mol_type = genomic DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttaaccaggcaggaactgctctgatctttgggaagggaaccaccttatcagtgagttccaat

SEQ ID NO: 42 - DNA, 336 bp, Homo sapiens
  source 1..336: mol_type = genomic DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcatggggacaggagatgaagctttctttggacaaggcaccagactcacagttgta

SEQ ID NO: 43 - protein, 12 aa, Homo sapiens
CAVNQAGTALIF

SEQ ID NO: 44 - protein, 12 aa, Homo sapiens
CASMGTGDEAFF

SEQ ID NO: 45 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 46 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 47 - protein, 269 aa, synthetic construct
  REGION 1..269: note = Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 307 aa, synthetic construct
  REGION 1..307: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASMGTGDEAFFGQGTRLTVVDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 282 aa, synthetic construct
  REGION 1..282: note = Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVNQAGTALIFGKGTTLSVSSNSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 50 - protein, 304 aa, synthetic construct
  REGION 1..304: note = Synthetic
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASMGTGDEAFFGQGTRLTVVDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

(19) United States
(12) Patent Application Publication
(10) Pub. No.: US 2024/0415886 A1
(43) Pub. Date: Dec. 19, 2024

(54) T CELL RECEPTOR RECOGNIZING R175H MUTATION IN P53 AND ITS APPLICATION

(71) Applicants: Yafei HOU, Mountain View, CA (US); David HOU, Mountain View, CA (US)

(72) Inventors: Yafei HOU, Mountain View, CA (US); David HOU, Mountain View, CA (US)

(21) Appl. No.: 18/701,607

(22) PCT Filed: Oct. 28, 2022
(86) PCT No.: PCT/US2022/078929

§ 371 Date: Apr. 15, 2024

Related U.S. Application Data
(60) Provisional application No. 63/273,372, filed on Oct. 29, 2021.

(51) Int. Cl. A61K 35/17; A61K 39/00; C07K 16/28; C07K 16/30; C12N 15/86

(52) U.S. Cl. CPC A61K 35/17; A61K 39/4611; A61K 39/4613; A61K 39/4632; A61K 39/464414; A61K 39/464451; C07K 16/2827; C07K 16/30; C12N 15/86; A61K 2239/11; C07K 2317/565; C12N 2740/15043

(57) ABSTRACT

This invention provides an isolated or purified T-cell receptor (TCR) having antigenic specificity for the mutated human p53R175H. The inventive TCR can recognize the HLA-A2 restricted mutant p53R175H peptide (HMTEVVRHC) but cannot recognize the wild-type p53R175 peptide (HMTEVVRRC). The inventive TCR can also be activated by tumor cells harboring a mutated human p53R175H in the context of HLA-A2. The invention further provides related polypeptides, proteins, nucleic acids, recombinant expression vectors, host cells, populations of cells and pharmaceutical compositions relating to the TCR of the invention. Methods of treating or preventing cancer in a mammal are further provided by the invention.

### CROSS-REFERENCE TO RELATED APPLICATIONS

The present application claims the priority benefit of International Patent Application No. PCT/US2022/078929, filed on Oct. 28, 2022, and claims the benefit of U.S. Provisional Patent Application No. 63/273,372, filed on Oct. 29, 2021, which are hereby incorporated herein by reference in their entireties.

### SEQUENCE LISTING

The instant application contains a Sequence Listing which has been submitted electronically in XML format and is hereby incorporated by reference in its entirety. Said XML copy, created on Oct. 22, 2022, is named ‘IMSYN001PCT-p53.xml’ and is 63,207 bytes in size.

### TECHNICAL FIELD

The embodiments of the present invention relate to peptides, proteins, nucleic acids, and cells for application in cancer immunotherapy. Some embodiments of the present invention relate to compositions and methods for the immunotherapy-based prevention or treatment of cancer with TP53 (p53) mutation utilizing T cell receptor designed to specifically recognize tumors expressing a mutant TP53 epitope presented by HLA-A*02 molecules, including HLA-A*02:01 molecules.

### BACKGROUND OF THE INVENTION

There is a general desire for new efficacious and safe cancer treatment options. Adoptive cell therapy (ACT) with tumor-infiltrating lymphocytes (TIL), or the genetically modified T cells has been proven to be one of the most promising immunotherapies against cancer (N Engl J Med 2017; 377:2545-2554). Adoptively transferring T cells generated from TILs or the T cells that are genetically modified to express the specific T cell receptor (TCR) against tumor antigens to the patients with solid tumors had shown clinical benefits (Adv Immunol. 2016; 130:279-94). Clinical trials of TCR based T therapy (TCR-T) for various solid tumors are currently being conducted at different phases (Technol Cancer Res Treat. 2019; 1-13). Studies revealed that neoantigen-specific T cells are crucial for clinical responses. Neoantigens are generated by the genomic codon alternations such as somatic mutations in tumor and could be presented by the major histocompatibility complex (MHC; also known as human leukocyte antigen (HLA) in humans) on the cell surface and recognized by the T lymphocytes (Science. 2007; 318:1108-1113). Adoptive T cell therapies targeting neoantigens is one of the most promising immunotherapy approaches to treat solid cancers. One challenge in translating neoantigen-targeted immunotherapies to patients with cancer is the unique neoantigen repertoire of each patient. There are few shared mutated targets among patients, even among patients with similar histologic cancer types. However, the identification of the therapeutic regiments targeting a shared immunogenic neoantigen would facilitate the development of therapies that could be more broadly applied to patients with cancer.

Mutated TP53 represents an ideal shared antigen target. In an assessment of four different pan-cancer sequencing studies, TP53 was found to be mutated in approximately 50% of sequenced cases, representing a broad range of cancer types, including 89% of lung, 72.7% of colorectal cancer, and 70.7% of esophageal cancer (Nat Med 2017; 23:703-13). Moreover, cancers with TP53 mutations frequently have “hotspot” mutations at amino acid positions R175, G245, R248, R249, R273, and R282. Thus, immunogenic agents that can specifically target those TP53 mutations have potential efficacy in the diagnosis, treatment and/or prophylaxis of cancer. Amino acid location R175 is one of the most frequently mutated residues and comprises approximately 5% of all identified mutations in TP53. Ninety-four percent result in a histidine substitution (Cancer Discov. 2012; 2(5):401-4). This specific hotspot mutation can be a therapeutic target for multiple cancer cellular pathologies. Further, HLAA*02 is present in approximately 16% of African Americans and 48% of Caucasian American patients, making it one of the most dominant MHC class I HLAs in the United States (Nucleic Acids Res. 2015; 43:784-8).

### BRIEF SUMMARY OF THE INVENTION

TCR-T therapy targeting the mutated p53 p.R175H p53 p.R175H epitope presented by HLA-A*02 can be applied to treat a large number of patients with cancer. The following embodiments and aspects thereof are described and illustrated in conjunction with systems, tools and methods which are meant to be exemplary and illustrative, not limiting in scope.

An embodiment of the invention provides an isolated or purified T cell receptor (TCR) comprising antigenic specificity for a mutated human p53^{R175H} peptide presented by MHC class I molecule. In some embodiments, the mutated p53^{R175H} peptide comprises the amino acid sequence: HMTEVVRHC (SEQ ID NO:1). In some embodiments, the MHC class I molecule is HLA-A*02. In some embodiments, the MHC class I molecule is HLA-A*02:01, comprising the amino acid sequence of SEQ ID NO:2.

An embodiment of the invention provides an isolated or purified T cell receptor (TCR) comprising antigenic specificity for a mutated human p53^{R175H}, wherein the TCR comprises a first and a second chains, each one of the first and second chains comprising first, second and third complementarity determining regions (CDRs). The third CDR (CDR3) of the first chain comprises the amino acid sequence: CAVNQAGTALI (SEQ ID NO: 3), and the third CDR (CDR3) of the second chain comprises the amino acid sequence: CASMGTGDEAF (SEQ ID NO: 4).

In an embodiment, the isolated or purified T cell receptor comprises a first chain comprising the amino acid sequence of a combination of a Homo sapiens TRAV22*01 and a Homo sapiens TRJ15*01 (SEQ ID NO:11). A junction region interposed between the TRAV22*01 region and the TRJ15*01 region comprises the amino acid sequence: CAVNQAGTALIF (SEQ ID NO: 43).

In an embodiment, the isolated or purified T cell receptor comprises a second chain comprising the amino acid sequence of a combination of a Homo sapiens TRBV02*01, a Homo sapiens TRJ1-1*01 and a Homo sapiens TRD1*01 (SEQ ID NO:12). A junction region interposed between the TRBV02*01 region and the TRJ1-1*01 region comprises the amino acid sequence: CASMGTGDEAFF (SEQ ID NO: 44).

In an embodiment, the isolated or purified T cell receptor comprises a first chain comprising a first CDR (CDR1) comprising the amino acid sequence: DSVNN (SEQ ID NO: 7).

In an embodiment, the isolated or purified T cell receptor comprises a first chain comprising a second CDR (CDR2) comprising the amino acid sequence: IPSGT (SEQ ID NO: 8).

In an embodiment, the isolated or purified T cell receptor comprises a second chain having a first CDR (CDR1) comprising the amino acid sequence: SNHLY (SEQ ID NO: 9).

In an embodiment, the isolated or purified T cell receptor comprises a second chain comprising a second CDR (CDR2) comprising the amino acid sequence: FYNNEI (SEQ ID NO: 10.

In an embodiment, the isolated or purified T cell receptor comprises a first chain having its first, second and third CDRs comprising the amino acid sequence of SEQ ID NO: 7, SEQ ID NO: 8 and SEQ ID NO: 3 respectively, and a second chain comprising its first, second and third CDRs comprising the amino acid sequence of SEQ ID NO: 9, SEQ ID NO: 10 and SEQ ID NO: 4 respectively. In this regard, the TCR, e.g., comprises the amino acid sequences of any one of SEQ ID NO: 11, SEQ ID NO: 12, SEQ ID NO: 23 and SEQ ID NO: 24.

In an embodiment, the isolated or purified T cell receptor comprises a first full-length chain, as shown in SEQ ID NO: 5, having a variable region comprising the amino acid sequence of SEQ ID NO: 11 and a murine constant region of alpha comprising the amino acid sequence of SEQ ID NO: 28. In another embodiment, the isolated or purified T cell receptor comprises a first full-length chain, as shown in SEQ ID NO: 47, comprising a variable region comprising the amino acid sequence of SEQ ID NO: 11 and a human constant region as shown in SEQ ID NO: 26. In another embodiment, the isolated or purified T cell receptor comprises a second full-length chain, as shown in SEQ ID NO: 49, comprising a variable region comprising the amino acid sequence of SEQ ID NO: 11 and a human constant region of delta chain as shown in SEQ ID NO:45.

In an embodiment, the isolated or purified T cell receptor comprises a second full-length chain, as shown in SEQ ID NO: 6, comprising a variable region comprising the amino acid sequence of SEQ ID NO: 12 and a murine constant region of beta chain comprising the amino acid sequence of SEQ ID NO: 27. In another embodiment, the isolated or purified T cell receptor comprises a second full-length chain, as shown in SEQ ID NO: 48, comprising a variable region comprising the amino acid sequence of SEQ ID NO: 12 and a human constant region of beta chain as shown in SEQ ID NO: 25. In another embodiment, the isolated or purified T cell receptor comprises a second full-length chain, as shown in SEQ ID NO: 50, comprising a variable region comprising the amino acid sequence of SEQ ID NO: 12 and a human constant region of gamma chain as shown in SEQ ID NO:46.

In an embodiment, the first chain of the inventive TCR comprises a combination of a variable region and a constant region, e.g., the first chain of the inventive TCR comprises a variable region comprising the amino acid sequence of SEQ ID NO: 11 and a beta constant region comprising the amino acid sequence of SEQ ID NO: 25, and the second chain of the inventive TCR comprises a variable region comprising the amino acid sequence of SEQ ID NO: 12 and a alpha constant region comprising the amino acid sequence of SEQ ID NO: 26.

In some embodiments, an isolated or purified polypeptide comprising a functional portion of the TCR comprising the amino acid sequence or sequences selected from a group consisting of SEQ ID NOs: 5, 6, 11, 12, 23, 24, 47, 48, 49, 50.

In some embodiments, a single polypeptide comprising or consisting of a variable region of the first chain (SEQ ID NO: 23) and a variable region of the second chain (SEQ ID NO: 24) of the isolated or purified T cell receptor of this invention, wherein the variable region of the first chain and the second chain of the TCR can be connected by a linker peptide, e.g., a flexible linker peptide, e.g., a (GlyGlyGlyGlySer)n linker.

In some embodiments, a protein comprising two separate polypeptides comprising the first and second chains of the isolated or purified T cell receptor of this invention.

In some embodiments, a TCR, polypeptide, or protein comprises substantial or significant sequence identity or similarity to the inventive TCR, polypeptide, or protein, and retains the biological activity of the TCR, polypeptide, or protein of which it is a variant, e.g., comprising antigenic specificity for a mutated human p53^{R175H} peptide (SEQ ID NO: 1) presented by HLA-A2 or to which the parent polypeptide or protein specifically binds, to a similar extent, the same extent, or to a higher extent, as the parent TCR, polypeptide, or protein. In this regard, a TCR, polypeptide, or protein can, for instance, be at least about 75%, 80%, 90%, 95% o, 96%, 97%, 98%, 99% or more identical in amino acid sequence to any one of the amino acid sequences, selecting from a group of SEQ ID NOs: 3, 4, 7, 8, 9, 10, 11, 12, 23, 24.

An embodiment of the invention provides an isolated or purified nucleic acid comprising a comprising a nucleotide sequence encoding any of the inventive TCRs, polypeptides, or proteins described herein. The nucleic acid can be a DNA or RNA, which can be single-stranded or double-stranded, synthesized or obtained (e.g., isolated and/or purified) from natural sources.

An embodiment of the invention provides an isolated or purified nucleic acid comprising, from 5′ to 3′, (a) a first nucleotide sequence and a second nucleotide sequence, or (b) a second nucleotide sequence and a first nucleotide sequence, wherein the first nucleotide sequence encodes any one of the amino acid sequence selected from a group of SEQ ID NOs: 5, 11, 24, 47, 49; and the second nucleotide sequence encodes any one of the amino acid sequence selected from a group of SEQ ID NOs: 6, 12, 23, 48, 50.

In an embodiment of the invention, the isolated or purified nucleic acid further comprises a third nucleotide acid sequence interposed between the first and second nucleotide sequence so that the first and second chains are cleaved into or expressed as two separate polypeptides. The suitable sequence can be a T2A, P2A, E2A, F2A or IRES sequence. In an embodiment of the invention, the isolated or purified nucleic acid encodes an amino acid sequence consisting of SEQ ID NO: 13.

In some embodiments of the invention, the isolated or purified nucleic acid molecule comprises the nucleotide sequence of any one of SEQ ID Nos: 16, 17 and 29-40.

In some embodiments, antigen binding domain comprising three CDRs of each of the first and second chains of the inventive TCR (SEQ ID NOs: 3, 7, 8 and SEQ ID NOs: 4, 9, 10 respectively) are configured to be expressed as a single polypeptide. The first and second chains are joined by a flexible linker to form an antigen binding domain specific for the p53^{R175H} peptide (SEQ ID NO: 1) in the context of HLA-A2. The antigen binding domain herein joined with a T cell costimulatory domain, e.g., a costimulatory domain from CD28, 4-1BB, CD27 or OX40 and a T cell activation singling domain, e.g., a ITAM domain from CD3, can form an antigen targeting agent that is specific for the p53^{R175H} peptide (SEQ ID NO: 1) in the context of HLA-A2.

In an embodiment of the invention, the isolated or purified TCR, polypeptide, or protein described in the invention can be expressed by a recombinant expression vector. The recombinant expression vector can be a plasmid, a circulate RNA, a single-stranded DNA, a vector comprising transposon or CRISPR-Cas9, or a virial vector e.g., lentiviral vector.

In some embodiments of the invention, an isolated or purified TCR, polypeptide, or protein is encoded by any of the nucleic acids or vectors described herein, or results from expression of any of the nucleic acids or vectors described herein in a cell.

Another embodiment of the invention provides a method of producing a host cell expressing a TCR that has antigenic specificity for the peptide of SEQ ID NO: 1 that is presented by HLA-A2.

In an embodiment of the invention, the host cell comprises a primary human lymphocyte, a human lymphocyte precursor or a stem cell that can differentiate to T cell. In an embodiment, the host cell is an immortalized human cell line.

In an embodiment, the host cell comprises a cell selected from the group consisting of a T cell, a natural killer T (NKT) cell, an invariant natural killer T (iNKT) cell, or a natural killer (NK) cell.

In an embodiment, the invention provides a pharmaceutical composition comprising any of the TCR or TCR variant, polypeptide, protein, nucleic acids, vectors and host cells described herein with antigen specificity for the mutated p53^{R175H} and a pharmaceutically acceptable carrier.

An aspect of the invention provides any of the TCRs, polypeptides, proteins, nucleic acids, recombinant vectors, host cells, and/or pharmaceutical compositions described herein, for using in treating cancer in a mammal. In an embodiment, the cancer includes but not is limited to cholangiocarcinoma, melanoma, colon cancer, rectal cancer, liver cancer, esophageal cancer, ovarian cancer, endometrial cancer, myeloma, leukemia, non-small cell lung cancer (NSCLC), glioblastoma, uterine cervical cancer, head and neck cancer, breast cancer, pancreatic cancer, sarcoma, or bladder cancer. Another aspect of the invention provides any of the inventive TCRs herein for use in preventing precancerous conditions and lesions in mammal that can develop into cancer. In an embodiment, the precancerous conditions and lesions that affect a variety of organ systems include but are not limited to colon polyps, cervical dysplasia, carcinoma in situ, monoclonal gammopathy of unknown significance, myelodysplastic syndromes or actinic keratosis. In another embodiment, the invention described herein is used in preventing cancer development from Li-Fraumeni Syndrome. In an embodiment, the cancer or precancerous cells carry HLA-A2 allele and the mutated p53^{R175H}. Another aspect of the invention provides the method involves sequencing a sample from the subject to verify the presence of p53 comprising a missense mutation of R175H and HLA typing to verify that the subject has an HLA-A*02 allele.

The embodiments herein with language “comprise,” “comprises” or “comprising” can be replaced with language “consist of”, “consists of” or “consisting of” in certain embodiments.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows the phenotypes of p53^{R175H} peptide specific CTLs and CTL clones that were generated from PBMCs of an HLA-A2+ healthy donor blood. FIG. 1A shows the representative flow cytometry plots from a cell culture that was re-stimulated with the mutated p53^{R175H} peptide (SEQ ID NO: 1) and expanded with cytokines in vitro. The cells were stained with APC labeled anti-CD8a and PE labeled HLA-A2/p53^{R175H} tetramer, then analyzed by flow cytometry (living gated on lymphocytes). Tetramer+ gate was set based on the control T cells. The frequency of CD8+ HLA-A2/p53^{R175H} tetramer+ T cells was displayed from the living gated lymphocytes. Square gated population represents CD8+ tetramer+ T cells that were sorted for T cell cloning. FIG. 1B shows representative flow cytometry plots from total fifty-eight T cell clones that were generated by FACS-sorting followed by in vitro stimulation with CD3/CD28 bead. Total seventeen CD8+ tetramer− T cell clones were obtained (represented by the upper-left plot) and forty-one CD8+ tetramer+ T cell clones were obtained (represented by the other plots).

FIG. 2A shows the schematic representation of a lentiviral vector for the expression of the inventive TCR. A nucleotide sequence (SEQ ID NO: 16) encoding a TCR specific for the mutated p53^{R175H} epitope described herein was cloned into MCS region of pCDH-EF1α-MCS-(PGK-GFP) bidirectional lentiviral vector. The nucleotide sequence of the recombinant lentivirus (pCDH-p53) comprises a nucleic acid that encodes the inventive TCR and is flanked with an EF1α promoter and WPRE region. A deletion in the enhancer of the U3 region (U3 del) ensures self-inactivation of the lentiviral construct. The p53^{R175H} peptide specific TCR described herein comprised a beta chain with the variable sequence (TCR-βV, SEQ ID NO: 12) and a murine constant sequence (Murine TCR-βC, SEQ ID NO: 27), an alpha chain with the variable sequence (TCR-αV, SEQ ID NO: 11) and a murine constant sequence (Murine TCR-αC, SEQ ID NO: 28), and a linker sequence (SEQ ID NO: 13). The bold underlined are the amino acid sequences of the junction region of the inventive TCR-αV between the AV22*01 and the TRJ15*01 (SEQ ID NO: 43) and the junction region of the inventive TCR-βV between the TRBV02*01 and the TRJ1-1*01 (SEQ ID NO: 44).

FIG. 2B shows that PBMCs transfected with the recombinant lentivirus encoding the inventive TCR could express the exogenous TCR that could be detected by HLA-A2/p53^{R175H} tetramer. GFP was used as the virus transfection marker. The transduced T cells were stained with APC labeled anti-CD8a antibody and PE labeled HLA-A2/p53^{R175H} tetramer and analyzed by flow cytometry. The left graph shows the percentage of GFP+ T cells in the living gated lymphocyte population, which represented T cells that were transduced with the recombinant lentivirus. The right graph shows the percentage of HLA-A2/p53^{R175H} tetramer+ T cells in the gated CD8-GFP+ T cell population (left plot) and in the gated CD8+GFP+ T cell population (right plot).

FIG. 2C shows that T cell lines transfected with the recombinant lentivirus encoding p53^{R175H} specific TCR could express the exogenous TCR that could be detected by HLA-A2/p53^{R175H} tetramer. J.RT3-T3.5 (JRT) cells that is derived from Jurkat cell line and does not express endogenous TCR and Jurkat cells that express exogenous human CD8 (Jurkat-CD8) were transduced with lentivirus encoding p53^{R175H} specific TCR. The transduced cells were stained with anti-CD8-APC and HLA-A2/p53^{R175H} tetramer-PE and analyzed by flow cytometry. The percentage of tetramer+ cells in the gated GFP+ JRT cells is displayed in the left plot. The percentage of tetramer+ cells in the gated GFP+ Jurkat-CD8 cells is displayed in the right plot.

FIG. 3 shows the functional analysis of primary T cells and T cell line that were transduced to express the inventive TCR specific for the mutated p53^{R175H} peptide (SEQ ID NO:1) in the context of HLA-A2. FIG. 3A shows the p53^{R175H} specific TCR expressed by JRT cells could recognize the mutated p53^{R175H} peptide presented by HLA-A2 on T2 cells. JRT cells transduced with the recombinant lentivirus (pCDH-p53) were co-cultured with T2 cells pulsed with 10× dilution series of the mutated p53^{R175H} peptide starting from 1000 ng/ml for 16 hours. The cells were stained with anti-CD69 antibodies to analyze the percentage of the activated CD69+ cells in the gated GFP+ cells by flow cytometry. X-axis is the concentration of the p53^{R175H} peptide, Y-axis is the percentage of CD69+ cells in the gated GFP+ JRT cells. FIG. 3B shows the inventive TCR expressed by primary T cells generated from PBMCs could recognize the mutated p53^{R175H} epitope peptide but not the wild-type p53^{R175} peptide (SEQ ID NO: 20) presented by HLA-A2 on T2 cells. PBMC cells transduced with the recombinant lentivirus (pCDH-p53) were co-cultured with T2 cells pulsed with 10× dilution series of the mutated p53^{R175H} peptide (square) starting from 1000 ng/ml or the wild type p53^{R175} peptide (triangle) starting from 10 ug/ml for 24 hours. The cells were stained with APC labeled anti-CD8a and PE labelled anti-CD137 antibodies to analyze the percentage of CD8+CD137+ cells in the gated GFP+ cells by flow cytometry. X-axis is the concentration of the p53^{R175H} peptide, Y-axis is the percentage of CD8+CD137+ cells in the gated GFP+ living lymphocytes.

FIG. 4 is a graph showing the functional analysis of primary T cells that express inventive TCR specific for the mutated p53^{R175H} peptide against tumor cell lines. FIG. 4A is a graph showing the plots of HLA-A2 expression on the target tumor cells. The cells were stained with anti-HLA-A2 antibody labelled with PE and analyzed by flow cytometry. The left graph shows HLA-A2 expression by three tumor cell lines. Light gray line was the negative control, medium gray line represented the tumor line that was not pre-treated with IFN-gamma and dark gray line represented the tumor cell line that was pre-treated with IFN-gamma at 200u/ml for 24 hours. The right graph shows HLA-A2 expression by the tumor cell line that was transduced to express exogenous HLA-A2. Light gray line was the negative control, medium gray line represented the tumor line that was transduced to express HLA-A2, and dark gray line represents the tumor cell line that was transduced to express HLA-A2. FIG. 4B is the graph showing that PBLs that were transduced to express the inventive TCR could recognize the HLA-A2+ tumor cells with the endogenous mutated p53^{R175H}. The activated PBMCs were transduced with the recombinant lentivirus (pCDH-p53) to express the inventive TCR specific the mutated p53^{R175H} peptide in the context of HLA-A2 and cocultured with different target cells, including tumor cell lines expressing both HLA-A2 and p53 protein with R175H mutation (KMS26, KLE, SKUT1-A2BM, SKUT1-A2, 293T-A2-mp53 and K562-A2-mp53), target cells expressing p53 protein with R175H mutation alone (SKUT1) or target cells expressing HLA-A2 alone (293T-A2, SKOV3-A2). 293T-A2, SKOV3-A2, SKUT1-A2 and SKOV3-A2 are cell lines transduced to express exogenously HLA-A2. SKUT1-A2BM is the cell line transduced to express exogenously HLA-A2 and beta2-microgluboine. 293T-A2-mp53 and K562-A2-mp53 are transduced to express exogenously both HLA-A2 and p53 protein with R175H mutation. The target cells were not treated (filled bar) or were treated with IFN-gamma at 200 ng/ml for 24 hours (dotted bar). Some mixed-cultures were treated with anti-HLA-ABC antibody at 2 ug/ml (opened bar). Using the transduced PBMCs alone as negative control. Using T2 cells pulsed with the mutated p53^{R175H} peptide (T2+mt-p53 peptide) at 100 ng/ml as positive control. After incubated for 24 hours the cells were stained with anti-CD8a-APC and anti-CD137-PE antibodies to analyze the percentage of CD8+CD137+ cells in the gated GFP+ living lymphocytes by flow cytometry. CD137 is a T cell activation marker. X-axis is the targets; Y-axis is the percentage of CD8+CD137+ cells in the gated GFP+ living lymphocytes.

### DETAILED DESCRIPTION

The following explains in detail the specific embodiments and references to the figures of the present disclosure, but it is not the limitation of the present invention. Based on the basic principles of the present disclosure, the modifications or improvements made by the technical personnel in the field are within the scope of the present invention as long as they comply with the basic principles of the present disclosure.

As used herein, the terms “CTLs” refers to CD8-positive cytotoxic T cells (CTLs). CD8+ CTLs are able recognize the antigen epitope presented by MHC class I molecules on the surface of target cells. Similarly, “CD4+ Th” refers to CD4-positive T help cells.

As used herein, the term “antigen” refers to molecules that can induce an immune response. The term “epitope” refers to the part of an antigen that can stimulate an immune response. For example, an epitope may be a peptide that is bound to MHC class I molecule to thereby form an MHC-I/peptide complex. The MHC-I/peptide complex can be selectively recognized by a specific T-cell receptor (TCR) of a cytotoxic T-cell.

As used herein, the term “T cell response” means the proliferation and activation of effector T cells when TCR of the effector T cells are activated by the cognate antigen. For example, T cell response of MHC class I restricted CTLs may include lysis of target cells, secretion of cytokines, or expression of effector molecules (e.g., CD69 and/or 4-1BB). The effector molecules herein can be used as biomarkers to indicate the T cell response to the cognate antigen stimulation. A TCR can be considered as being activated or having “antigenic specificity” for a mutated target if at least twice as many of the numbers of T cells expressing the TCR express effector molecules (e.g., CD69 and/or 4-1BB) upon co-culture with target cells that express the mutated epitope peptide presented by HAL class I molecule.

The inventive TCRs, polypeptides, proteins, nucleic acids, recombinant expression vectors, and host cells (including populations thereof), can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be or be greater than 60%, 70%, 80%, 90%, 95%, or can be 100%.

As used herein, the term “variant” means in the context of proteins or polypeptides, one or two or more of the amino acid residues are replaced with other amino acid residues, while the variant retains substantially the same biological function as the unaltered protein.

The terms “treat”, “treating” and “treatment” refer to an approach for obtaining desired clinical results. Desired clinical results can include, but are not limited to, reduction or alleviation of at least one symptom of a disease. For example, treatment can be diminishment of at least one symptom of disease, diminishment of extent of disease, stabilization of disease state, prevention of spread of disease, delay or slowing of disease progression, palliation of disease, diminishment of disease reoccurrence, remission of disease, prolonging survival with disease, or complete eradication of disease. The terms “prevent”, “prevention” and “preventing” refer to a procedure through which individuals, particularly those with risk factor for a disease, e.g., with a missense mutation in p53 gene, are treated in order to prevent a disease from occurring or progressing.

Tumor Protein p53 (also referred to as “TP53” or “p53”) acts as a tumor suppressor by, for example, regulating cell division. The p53 protein is located in the nucleus of the cell, where it binds directly to DNA. Wild type p53 (hereinafter, WT p53, SEQ ID NO: 18). Mutations of p53 are defined herein by reference to the amino acid sequence of full-length WT p53 and are described herein by reference to the amino acid residue present at a particular position, followed by the position number, followed by the amino acid with which that residue has been replaced in the particular mutation under discussion. P53 comprises nine known splice variants. The p53 mutations described herein are conserved over all nine p53 splice variants. The mutations refer to a replacement of an amino acid residue in the amino acid sequence of a particular splice variant of p53 corresponding to the indicated position of the 393-amino acid sequence of SEQ ID NO: 18 with the understanding that the actual positions in the splice variant may be different.

As used herein, the term “p53^{R175H}” or “R175H” refers to missense mutants at p53 codon position 175 wherein the wild type of arginine residue is mutated to a histidine residue (SEQ ID NO: 19). The term “p53^{R175H} peptide” or “p53^{R175H} epitope peptide” refers to a peptide containing a missense mutation originally at p53 codon position 175 wherein the wild type of arginine residue is mutated to a histidine residue (SEQ ID NO: 1). The term “wild type p53^{R175} peptide” or “p53^{R175} epitope peptide” refers to a peptide derived from WT p53 wherein an arginine residue is at p53 codon position 175.

As used herein, the term “T cell receptor” or “TCR” refers to functional portions and functional variants of the T cell receptor, unless specified otherwise. A TCR comprises two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The TCR of the invention provides many advantages, including when expressed by effector cells used for adoptive cell transfer. Since the mutated p53 is expressed by cancer cells and are not expressed by normal cells, the effector cells that are modified to express the TCR of the invention specifically destroy cancer cells while minimizing or eliminating the destruction of normal cells.

In an embodiment of the invention, the TCR comprises antigenic specificity for human p53 with a mutation at position 175, as defined by SEQ ID NO: 19. The p53 mutation at position 175 may be any missense mutation. In an embodiment of the invention, the TCR comprises antigenic specificity for human p53 with the R175H mutation. In other embodiments, the TCR comprises antigenic specificity for human p53 with a R175G mutation, a R175L mutation, a R175A mutation, a R175C mutation or a R175D mutation. The mutated p53 herein refers to any mutated p53 protein, polypeptide or peptide. In a preferred embodiment of the invention, the TCR comprises antigenic specificity for a mutated p53^{R175H} peptide comprising or consisting of the amino acid sequence of HMTEVVRHC (SEQ ID NO: 1). The TCR cannot recognize the counterpart wild type p53 peptide comprising or consisting of the amino acid sequence of HMTEVVRRC (SEQ ID NO: 20). The length of the p53^{R175H} peptides can vary so long as the peptides can be recognized by the TCR of the invention, in some embodiments, may comprise one to five additional amino acids on the amino or carboxyl terminus of SEQ ID NO: 1.

In an embodiment of the invention, the TCR may be able to recognize a mutated p53 peptide presented by HLA (human leukocyte antigen) molecule. In this regard, the TCR may recognize the mutant p53^{R175H} peptide within the context of an HLA Class I molecule. In an embodiment of the invention, the HLA Class I molecule is an HLA-A molecule. The HLA-A molecule α chain, beta2-microglobulin and the mutant p53^{R175H} peptide form HLA/peptide complex that is expressed on surface of target cells and is specifically recognized by the inventive TCR. In an embodiment of the invention, the HLA Class I molecule is an HLA-A2 molecule. The HLA-A2 molecule may be any HLA-A2 molecule or HLA-A2 supertypes, including, but are not limited to, HLA-A*02:01, HLA-A*02:02, HLA-A*02:03, HLA-A*02:05, HLA-A*02:06, HLA-A*02:07, or HLA-A*02: 11, HLA-A*68:02. Preferably, the HLA Class I molecule is an HLA-A*02:01 molecule.

The TCR of the invention can recognize the mutated p53^{R175H} peptide in the context of HLA-A2 with specificity. A TCR may be considered to have antigen specificity for the mutated p53 if primary T cells or T cell lines that express the TCR upregulate expression of T cell activation markers, e.g., CD137 or CD69 (activated T cells) upon co-culture with HLA-A2 positive target cells pulsed with low concentration of the mutated p53 peptide (e.g., about 0.5 ng/mL, 1 ng/mL, 5 ng/mL, 10 ng/ml, 50 ng/ml, 100 ng/ml, 1000 ng/ml or a range defined by any two of the foregoing values). The frequency of the activated T cells expressing a T cell activation marker should be at least twice as much as compared to the amount of a negative control. Additionally, a TCR may be considered to have antigen specificity for mutated p53^{R175H} if T cells that express the TCR upregulate expression of T cell activation markers upon co-culture with HLA-A2 positive target cells that endogenously express mutated p53^{R175H} protein comprising an epitope peptide with the sequence of SEQ ID NO: 1, or with the HLA-A2 positive target cell that is transduced with a nucleic acid to express the mutated p53^{R175H}. The expression of T cell activation markers can be measured by, for example, flow cytometry after stimulation with target cells described above. Alternatively, a TCR may be considered to have antigen specificity for mutated p53^{R175H} if T cells expressing the inventive TCR produce at least twice as much IFN-gamma or GM-CSF upon co-culture with HLA-A2 positive target cells pulsed with the mutated p53 peptide, or HLA-A2 positive target cells endogenously expressing the mutated p53^{R175H}, or target cells that are transduced with a nucleotide acid to express the mutated p53^{R175H} as compared to the amount of IFN-γ or GM-CSF produced by a negative control. IFN-γ or GM-CSF secretion may be measured by methods known in the art such as, for example, enzyme-linked immunosorbent assay (ELISA) or enzyme-linked immunospot (ELISOT) assay.

In an embodiment of the invention, the inventive TCR comprises two polypeptide chains, each of which comprises a variable region comprising a complementarity determining region CDR 1, a CDR2, and a CDR3. In an embodiment of the invention, the TCR comprises a first polypeptide chain comprising an α chain CDR1 (CDR1α), an α chain CDR2 (CDR2α), and an α chain CDR3 (CDR3α), and a second polypeptide chain comprising a β chain CDR1 (CDR1β), a β chain CDR2 (CDR2β), and α chain CDR3 (CDR3β). In an embodiment of the invention, the inventive TCR comprises the amino acid sequences of SEQ ID NOs: 7, 8, 3, 9, 10, 4 that are the six CDR regions of the TCR specific to the mutated p53^{R175H}, corresponding to the CDR1α, CDR2α, CDR3α, CDR1β, CDR2β, and CDR3β of the inventive TCR, respectively.

In an embodiment of the invention, the inventive TCR comprises an α chain variable region comprising a CDR1α, a CDR2α and a CDR3α (SEQ ID NO: 7, 8, 3 respectively), or a β chain variable region comprising a CDR1β, a CDR2β, and a CDR3β (SEQ ID NO: 9, 10, 4 respectively). In an embodiment, the inventive TCR comprises both amino acid sequences of SEQ ID NO: 11 and 12, the two amino acid sequences corresponding to the variable region of the α chain and the variable region of the β chain of the inventive TCR respectively. The inventive TCR described herein has antigen specificity for the mutated p53^{R175H}.

In an embodiment of the invention, the inventive TCR comprises an α chain or a β chain comprising a native signal peptide (SEQ ID NO: 22) or a native signal peptide (SEQ ID NO: 21) respectively. In another embodiment, the inventive TCR comprises an α chain or a β chain comprising a signal peptide of another secreted protein or membrane bound protein. In another embodiment, the inventive TCR comprises the mature Vu chain without a signal peptide (SEQ ID NO: 24) or a mature Vβ chain without a signal peptide (SEQ ID NO: 23).

In an embodiment of the invention, the TCR comprises a constant region. The TCR constant region may be derived from other species such as, e.g., human or mouse. In an embodiment, the TCRs comprise a human α chain constant region (SEQ ID NO:26) and a human β chain constant region (SEQ ID NO:25). In an embodiment, the TCRs comprise a murine α chain constant region (SEQ ID NO:28) and a murine β chain constant region (SEQ ID NO:27). An advantage of using murine constant region is to limit mispairing of the exogenous TCR chains, e.g., alpha and beta chains, with the endogenous TCR chains, e.g., alpha and beta chains. The inventive TCR can comprise a chain comprising an α chain variable region and a β chain constant region, or a chain comprising a β chain variable region and a α chain constant region, or both two chains comprising an α chain variable region and a β chain constant region and a β chain variable region and a α chain constant region.

In some embodiments of the invention, TCRs can be TCR variants of the inventive parent TCR. A TCR variant described herein, refers to a TCR having substantial or significant sequence identity or similarity to a parent TCR, and may retains the biological activity of the parent TCR, such as e.g., having antigen specificity for p53^{R175H}, or being able to specifically react to the mutated p53^{R175H}, to a similar extent, the same extent, or to a higher extent, as the parent TCR. Compared to the parent TCR and its six CDR regions including CDR1α, CDR2α, CDR3α, CDR1β, CDR2β, and CDR3β (SEQ ID NOs: 7, 8, 3, 9, 10, 4 respectively), an inventive TCR variant and its six CDR regions can, for instance, be at least about 75%, at least about 80%, at least about 90%, at least about 95%, at least about 96%, at least about 97%, at least about 98%, at least about 99% or more identical in amino acid sequence to the parent TCR and each of its six CDR regions, respectively.

An inventive TCR variant described herein can, e.g., comprise the amino acid sequence of the parent TCR and its six CDR regions including CDR1α, CDR2α, CDR3α, CDR1β, CDR2β, and CDR3β (SEQ ID NOs: 7, 8, 3, 9, 10, 4 respectively) with at least one conservative amino acid substitution. Conservative amino acid substitutions are known in the art and include amino acid substitutions in which one amino acid having certain physical and/or chemical properties is exchanged for another amino acid that has the same chemical or physical properties.

An inventive TCR variant described herein can, e.g., comprise an α chain with the three CDRs (SEQ ID NOs: 7, 8, 3) that comprise at least one substituted amino acid, and a β chain with the three CDRs (SEQ ID NOs: 9, 10, 4) that comprise at least one substituted amino acid. The biological activities of such TCR variants may be increased compared to the parent TCR. In an embodiment, the TCR variant comprise any one of α chains, β chains or both with a substituted CDRs, e.g., an α chains comprises a substituted CDR1α with any one of amino acid sequences: XSVNN, DXVNN, DSXNN, DSVXN or DSVNX; a substituted CDR2α with any one of amino acid sequences: XPSGT, IXSGT, IPXGT, IPSXT or IPSGX; or a substituted CDR3α with any one of amino acid sequences: XVNQAGTALI, AXNQAGTALI, AVXQAGTALI, AVNXAGTALI, AVNQXGTALI, AVNQAXTALI, AVNQAGXALI, AVNQAGTXLI, AVNQAGTAXI or AVNQAGTALX. A β chain comprises a substituted CDR1β with any one of amino acid sequences: XNHLY, SNHLY, SNXLY, SNHXY or SNHLX; a substituted CDR2β with any one of amino acid sequences: XYNNEI, FXNNEI, FYXNEI, FYNXEI, FYNNXI or FYNNEX; or a substituted CDR3β with any one of amino acid sequences: XSMGTGDEAF, AXMGTGDEAF, ASXGTGDEAF, ASMXTGDEAF, ASMGXGDEAF, ASMGTXDEAF, ASMGTGXEAF, ASMGTGDXAF, ASMGTGDEXF or ASMGTGDEAX. Wherein X is arginine, asparagine, asparatic acid, cysteine, glutamic acid, glutamine, glycine, histidine, isoleucine, leucine, lysine, methionine, phenylalanine, proline, serine, threonine, tryptophan, tyrosine, or valine. Each of the substituted CDRs of the TCR variants described herein does not comprise the parent CDR1α, CDR2α, CDR3α, CDR1β, CDR2β or CDR3β counterpart amino acid sequences as shown in SEQ ID NO: 7, 8, 3, 9, 10, 4 respectively.

In some embodiments, the inventive TCR or TCR variants can be a polypeptide or a protein (e.g., a molecule comprising one or more polypeptide chains) comprising a functional portion of any of the inventive TCR CDRs or any part or fragment of the TCR of the invention described in SEQ ID NOs: 3-12, 21-28 and 43-50, e.g., the functional portion can comprise about 10%, about 25%, about 30%, about 50%, about 68%, about 80%, about 90%, about 95%, or more, of the parent TCR CDRs. The functional portion means having antigen specificity for the mutated p53^{R175H}, or detect, treat, or prevent cancer, to a similar extent, the same extent, or to a higher extent, as the parent TCR. Suitable variants can be a single chain polypeptide comprising the functional portion domains of the inventive T-cell receptor, e.g., comprising an α chain variable region of the inventive TCR, a β chain variable region of the inventive TCR or both. In an embodiment, the single chain polypeptide described herein can be made as part of a soluble molecule that has the biological activity such as e.g., having antigen specificity for the mutated p53^{R175H} and being able to deliver effector molecules, e.g., delivering anti-CD3 scFv chain as a bispecific T cell engager, or delivering a cancer cytotoxic payload as known in the art to tumor cells with the mutated p53^{R175H} as a drug conjugate. In another embodiment, the single chain polypeptide described herein can be inserted into a chimeric antigen receptor (CAR) construct in place of the typical antibody scFv fragment so that the single chain polypeptide described herein interacts with the signaling domain of the CAR construct to cause the desired cytotoxic activity towards cancer cells. The signaling domain including a suitable co-stimulatory domain, (e.g., CD8, CD27, CD28, 4-1 BB, ICOS, 0X40, MYD88, IL1-R1, CD70), as well as any other domains, e.g., a CD3ζ or a CD3ε segment with ITAM domains, intended to enhance the characteristics of the CAR construct.

In some embodiments, the inventive TCR or TCR variants can be modified to be such as e.g., glycosylated, amidated, carboxylated, phosphorylated by methods known in the art, e.g., a disulfide bridge, or polymerized, or conjugated.

The TCR or TCR variants of the invention can be synthetic, recombinant, isolated, and/or purified polypeptide, and/or protein that is synthesized by methods known in the art or can be recombinantly produced using standard recombinant methods known in the art or can be commercially synthesized by companies.

Some embodiments of the invention relate to nucleic acids, recombinant vectors, host cells, populations of cells and pharmaceutical compositions encoding, incorporating or relating to the inventive TCR or TCR variant described above.

An embodiment of the invention provides a nucleic acid comprising a nucleotide sequence encoding a polypeptide comprising any of inventive TCR, TCR variant or its CDRs described herein. “Nucleic acid,” as used herein generally means a polymer of DNA or RNA, which can be single-stranded or double-stranded, synthesized or obtained (e.g., isolated and/or purified) from natural sources. The nucleic acids described herein can be recombinant. the term “recombinant” refers to the molecules that are constructed outside living cells by joining natural or synthetic nucleic acid segments to nucleic acid molecules that can replicate in a living cell.

In an embodiment of the invention, the inventive nucleic acid may comprise any one of the nucleotide sequence of CDRs of the inventive TCR as shown in the nucleotide sequence of SEQ ID NO: 29; the nucleotide sequence of SEQ ID NO: 30; the nucleotide sequence of SEQ ID NO: 31; the nucleotide sequence of SEQ ID NO: 32; the nucleotide sequence of SEQ ID NO: 33; the nucleotide sequence of SEQ ID NO: 34. In one embodiment, the nucleic acid comprises the nucleotide sequences of all of SEQ ID NOs: 29-31; all of SEQ ID NOs: 32-34; all of SEQ ID NOs: 29-34. In an embodiment of the invention, the nucleic acid may comprise the nucleotide sequence of SEQ ID NO: 35; SEQ ID NO: 36; or both SEQ ID NOs: 35 and 36. In an embodiment of the invention, the nucleic acid may comprise the nucleotide sequence of SEQ ID NO: 41, SEQ ID NO: 42, or both of SEQ ID NOs: 41 and 42. In another embodiment of the invention, the nucleic acid can encode a full-length TCR with a constant region derived from human and comprise the nucleotide sequence of SEQ ID NO: 37, SEQ ID NO: 38, or both of SEQ ID NOs: 37 and 38. In another embodiment of the invention, the nucleic acid may comprise the nucleotide sequence encoding a full-length TCR with a constant region derived from other species such as, e.g., mouse. In this regard, the nucleic acid may comprise the nucleotide sequence of SEQ ID NO 39; SEQ ID NO 40; or both of SEQ ID NOs: 39 and 40.

In some embodiments of the invention, the inventive nucleic acid may comprise the nucleotide sequence encoding a linker polypeptide such as e.g., a skipping sequence or a sequence allowing initiation of translation at a site other than the 5′ end of an mRNA molecule, for example, a T2A, P2A, E2A, F2A or IRES sequence; or any other sequence that allows two distinct polypeptides to be translated from a single mRNA, e.g., a furin cleavage sequence with the minimal cleavage site Arg-X-X-Arg, or the like. A cleavage sequence interposed between the first and second chains of the inventive TCR, so that the first and second chains will be expressed as a single polypeptide and then cleaved or translated into two separate polypeptides. In another embodiment, the nucleic acid may comprise the nucleotide sequence encoding a linker polypeptide as shown in SEQ ID NO 13.

In an embodiment of the invention, the inventive nucleic acid comprises a codon-optimized nucleotide sequence encoding any of the TCR or TCR variant and its CDRs described herein. Codon optimization of the nucleotide sequence may involve substituting a native codon for another codon that encodes the same amino acid but can be translated more efficiently in host cells.

In an embodiment of the invention, the inventive nucleic acid comprising a nucleotide sequence that is at least about 60% or more, e.g., about 70%, about 80%, about 90%, about 91%, about 92%, about 93%, about 94%, about 95%, about 96%, about 97%, about 98%, or about 99% identical to any of the inventive nucleic acids encoding any of the TCR or TCR variant and its CDRs described herein.

In an embodiment of the invention, the nucleic acid comprising a nucleotide sequence which is complementary to the nucleotide sequence of any of the inventive nucleic acids encoding any of the TCR or TCR variant and its CDRs described herein.

The nucleic acids of the invention can be incorporated into a recombinant expression vector. The inventive recombinant expression vector herein comprises any of the nucleic acids encoding any of the TCR or TCR variant and its CDRs described herein. The inventive recombinant expression vectors can comprise any type of nucleic acids, including, but not limited to DNA and RNA (e.g., mRNA), which can be single-stranded or double-stranded, synthesized or obtained in part from natural sources, and which can contain natural, non-natural, or altered nucleotides. The inventive recombinant expression vectors can be any suitable recombinant expression vector and can be used to transform or transfect any suitable host cell, including, but not limited to, plasmid or virus. The viral vectors include, but are not limited to e.g., retroviral vectors (derived from Moloney murine leukemia Virus), lentiviral vectors (derived from human immunodeficiency type I virus (HIV)). The recombinant expression vector comprises regulatory sequences, such as transcription and translation initiation and termination codons, which are specific to the type of host cell. The recombinant expression vectors can be designed for either transient expression, for stable expression, or for both. Also, the recombinant expression vectors can be made for constitutive expression or for inducible expression. In an aspect, the said recombinant expression vectors comprising the TCR genes of the invention can be produced with the conventional recombinant technology in the art.

In some embodiments, the recombinant expression vectors comprise a nucleotide sequence encoding other functional molecules in addition to amino acid sequences of the inventive TCR, TCR variant and its CDRs described herein. In one embodiment, the other functional molecule is fluorescent proteins (such as e.g., GFP proteins) for the tracking of the T cells in vivo. In another embodiment, the recombinant expression vectors comprise a suicide gene to improve the safety of the adoptive T cell therapy. The suicide gene is a genetically encoded molecule that allows selective destruction of adoptively transferred cells (Front. Pharmacol., 2014; 5(254):1-22). The suicide genes encoded molecules include, e.g., herpes simplex virus thymidine kinase, ganciclovir, cytosine deaminase, 5-fluorocytosin, 5-fluorouracil, inducible FAS, inducible Caspase9, truncated CD20, EGFR, c-myc or RQR8. The nucleotide sequence encoding suicide gene and the nucleotide sequence encoding the inventive TCR are controlled independently by the different promoters; or by the same promoter while the TCR gene and the suicide gene are connected with the self-cleaving linker peptide described above.

In some embodiments, the expression of the inventive TCR, including TCR variants, and the other functional molecule can be driven by two different promoters. The promotors include strong promoter, weak promoter, constitutive promoter, inducible promoter, tissue-specific promoter, or differentiation-specific promoter. The promoter can be from a viral source or a non-viral source (e.g., eukaryotic promoter), such as PGK1 promoter, EF-1a promoter, CMV immediate early enhancer and promoter, SV40 promoter, Ubc promoter, CAG Promoter, TRE promoter, CamKIIa promoter, human beta actin promoter. In some embodiments, when two promoters drive two genes, the dual promoters are arranged in the opposite orientation or in the same orientations.

An embodiment of the invention further provides a host cell composing any of the inventive TCR or TCR variant, polypeptide, protein, or nucleic acid described herein. The host cell can be a eukaryotic cell, e.g., animal cell or can be a prokaryotic cell, e.g., bacteria cell. The host cell can be a cultured cell or a primary cell, e.g., isolated directly from an organism, e.g., a human. The host cells herein can be used to amplify the gene vectors (e.g., E. coli) or to produce recombinant TCR polypeptides or proteins (e.g., VERO, COS, or HEK293). Most preferably, the host cell is human cell, such as e.g., human peripheral blood lymphocyte (PBL) or a peripheral blood mononuclear cell (PBMC). More preferably, the host cell is T cell.

In some embodiments, the T cells described herein can be any T cells, including primary T cells, T cells from T cell lines, or T cells that are differentiated from T cell precursors or stem cells (e.g., hematopoietic stem cells or induced pluripotent stem cells). The T cells can be obtained from a variety of resources, including e.g., blood, bone marrow, lymph node, the thymus, or tumor tissues or fluids. The T cell herein can be any type of T cell, including e.g., CD8+ T cell, CD4+ T cell, alpha/beta T cell, gamma/delta T cell, NKT cell, naïve T cell, memory T cell and the like.

According to the usual method of in vitro culture of mammalian cells, T cells are cultured and expanded under appropriate culture conditions. For example, the cells can be passaged when they reach more than 70% confluence, and the culture medium is usually replaced with fresh culture medium in 2 to 3 days. Use directly when the cells reach a certain number or freeze them according to the protocols in the art. The in vitro culture time can be within 24 hours, or it can be as long as 14 days or longer. The frozen cells can be used in the next step after thawing.

In one embodiment, the T cells can be cultured in vitro for a few hours to 14 days, or any number of hours in between. T cell culture conditions include the use of basic culture media, including but not limited to RPMI 1640, AIM-V, DMEM, MEM, a-MEM, F-12, X-Vivo 15 and X-Vivo. Other conditions required for cell survival and proliferation include but are not limited to the use of serum (human or fetal bovine serum), interleukin-2 (IL-2), insulin, IFN-γ, IL-4, IL-7, GM-CSF, IL-10, IL-12, IL-15, IL-21, TGF-beta and TNF-α, other culture additives (including amino acids, sodium pyruvate, vitamin C, 2-mercaptoethanol, growth hormone, growth factor). The T cells can be placed in appropriate culture conditions, for example, the temperature can be 37° C., 32° C., 30° C., or room temperature, and the air condition can be, for example, air containing 5% CO2.

The inventive TCR or TCR variant including any polypeptides, proteins, nucleic acids and recombinant expression vectors, and the host cells described herein can be formulated into a composition, such as a pharmaceutical composition. In an embodiment, the invention provides a pharmaceutical composition comprising any of the inventive TCR or TCR variant with antigen specificity for the mutated p53^{R175H} and a pharmaceutically acceptable carrier. In an embodiment, the inventive pharmaceutical composition can comprise the inventive TCR or TCR variant including any polypeptides, proteins, nucleic acids and recombinant expression vectors, and the host cells described herein in combination with another pharmaceutically active agent(s) or drug(s), such as a chemotherapeutic agent, an immune modulator agent (e.g., immune checkpoint blockade, cytokine, or the like), or the other agent such as therapeutic antibody or vaccine.

Methods for preparing a pharmaceutical composition are known or in the art. It is preferred that the pharmaceutically acceptable carrier be one which has no detrimental side effects or toxicity under the conditions of use. The technical personnel in the field can understand that the therapeutic agents of the invention may also comprise the druggable excipients and additives, including pharmaceutical or physiological vehicles, excipients, diluents (including normal saline or phosphate buffered saline); the additives include carbohydrates, lipids, peptides, amino acids, antioxidants, adjuvants, preservation agents and others known in the field.

The inventive pharmaceutical composition can be formulated and given through the routes as following: the intraarterial, intravenous, subcutaneous, intracutaneous, intra-tumoral, intra-lymphatic, intrathecal, intra-cerebrospinal, intra-bone marrow, intra-muscular or intra-peritoneal administration.

In an embodiment, the amount or dose of the inventive pharmaceutical compositions described herein (e.g., TCR or TCR variant, polypeptides, or proteins, nucleic acids, recombinant expression vectors, host cells, populations of cells) administered to the subject (e.g., human) should be sufficient to effect, e.g., a therapeutic or prophylactic response, in the subject over a reasonable time frame. The dose will be determined by the efficacy of the particular inventive pharmaceutical compositions, described herein and the condition of the subject, e.g., the body weight of the treated subject. The dose of the inventive pharmaceutical compositions will be determined by the existence, nature and extent of any adverse side effects that might accompany the administration of a particular inventive pharmaceutical compositions. Typically, the attending physician will decide the dosage of the inventive TCR pharmaceutical compositions with which to treat each individual patient, taking into consideration a variety of factors, such as age, body weight, general health, diet, sex, inventive pharmaceutical compositions to be administered, route of administration, and the severity of the cancer being treated. In an embodiment in which the inventive pharmaceutical composition is a population of cells (e.g., T cells comprising the inventive TCR or TCR variant), the number of cells administered per infusion may vary, e.g., from about 1×10⁶ to about 1×10¹² cells or more. In certain embodiments, fewer than 1×10⁶ cells may be administered.

In an embodiment, the inventive TCR or TCR variant including any polypeptides, proteins, nucleic acids and recombinant expression vectors, the host cells and the pharmaceutical composition described herein can be used in methods of treating or preventing cancer. In this regard, the invention provides a method of treating or preventing cancer in a mammal, comprising administering to the mammal any of the inventive TCR or TCR variant including any polypeptides, proteins, nucleic acids and recombinant expression vectors, the host cells and the pharmaceutical composition described herein, in an amount effective to treat or prevent cancer in the mammal.

The tumors and/or cancers described herein include, e.g., breast cancer, head and neck cancer, glioblastoma, synoviosarcoma, kidney cancer, sarcoma, melanoma, lung cancer, esophageal cancer, colon cancer, rectal cancer, brain cancer, liver cancer, bone cancer, choriocarcinoma, neuroendocrine tumor, Pheochromocytoma, Prolactinoma, von Hippel-Lindau disease, Zollinger-Ellison syndrome, anal cancer, Cholangiocarcinoma, bladder cancer, urethral cancer, glioma, neuroblastoma, meningioma, spinal cord tumor, Bone tumor, chondrosarcoma, Ewing's sarcoma, cancer of unknown primary site, Carcinoid tumor, mesenchymal tumors, Paget's disease, cervical cancer, gallbladder cancer, eye cancer, Kaposi sarcoma, prostate cancer, testicular cancer, skin squamous cell carcinoma, mesothelioma, Multiple myeloma, ovarian cancer, pancreatic cancer, penile cancer, pituitary carcinoma, soft tissue sarcoma, retinoblastoma, intestinal tumor, stomach/gastric cancer, thymus carcinoma, gestational trophoblastic neoplasia, endometrial cancer, vaginal cancer, vulvar cancer, mycosis fungoides, insulinoma, Cardiac Sarcoma, Meningeal carcinomatosis, primary peritoneal carcinoma and malignant pleural mesothelioma.

In one embodiment, the cancer is a cancer which expresses mutated p53. The cancer expresses p53 with a mutation at positions 175, more preferably, with the mutated p53^{R175H}. In another embodiment, the HLA-I type of human that is subjected to be treated with the inventive TCR is HLA-A2 homozygosity or heterozygosity. More preferably, the HLA-I type of the treated subject is HLA-A*0201.

The embodiments herein with language “comprise,” “comprises” or “comprising” can be replaced with language “consist of”, “consists of” or “consisting of” in certain embodiments.

### EXAMPLES

The following examples further illustrate the invention but, of course, should not be construed as in any way limiting its scope. Unless otherwise specified, the experimental methods used in the following examples are performed using the experimental procedures, operations, materials, and conditions that are understood and routinely performed by technical personals in the art. For instance, the recombinant plasmids and viral vectors, or polypeptides and proteins can be recombinantly produced using the nucleic acids described herein using standard recombinant methods (Green and Sambrook, Molecular Cloning: A Laboratory Manual, 4th ed., Cold Spring Harbor Press, Cold Spring Harbor, NY, 2012).

The following materials and methods were employed for the experiments described in Examples 1-5.

### Materials and Methods

### 1. Cell Lines:

The cell line used to prepare lentivirus particles is 293T cell (ATCC CRL3216). The antigen presenting cell line is 174×CEM.T2 cell (T2, ATCC CRL-1992). The cell line for exogenous TCR gene expression and functional analysis is J.RT3-T3.5 (JRT cells, ATCC TIB-153). Tumor cell lines that were used as target cells include: human myeloma cell line KMS26 (JCRB JCRB 1187), human uterus adenocarcinoma cell line KLE (ATCC CRL-1622), human ovarian cancer cell line SKOV3 (ATCC HTB-77), human uterine leiomyosarcoma cell line SKUT1 (ATCC HTB-114), and human myelogenous leukemia cell line K562 (ATCC CCL 243). The SKOV3, SKUT1-A2, 293T-A2 and K562-A2 cell lines that are stably expressing exogenous HLA-A2 ware generated by transfecting these cell lines with a recombinant lentiviral vector (System Biosciences CD811A-A2) encoding HLA-A*0201. SKUT1-A2BM cell line was generated by transfecting SKUT1 cells with a lentiviral vector encoding both HLA-A*0201 and beta-2 macroglobulin (CD811A-A2BM). 293T-A2-mp53 and K562-A2-mp53 cell lines were generated by transfecting 293T-A2 and K562-A2 cells with a recombinant pCDNA3.3 plasmid (Thermo Fisher K830001) encoding the mutated p53^{R175H} protein (SEQ ID NO: 19).

### 2. Cell Culture Medium:

KLE, SKUT1, 293T and their transduced cells were cultured in Dulbecco's Modified Eagle Medium (DMEM) with higher glucose (VWR cat #VWRL0101-0500) supplemented with 10% Fetal Bovine Serum, 2 mM L-glutamine. The other cells lines are cultured in RPMI-1640 complete medium (Lonza, cat #12-115F) supplemented with Fetal Bovine Serum (ATCC 30-2020), 2 mmol/L L-glutamic acid, 1× Essential Amino Acids 50× (Invitrogen 11130-051), 1× Streptomycin/Penicillin 100× (Invitrogen 15140-122), 1× Sodium pyruvate 100× (Invitrogen 11360-070), and 1×2-mercaptoethanol 1000× (Thermo fisher 21985023).

### 3. Peripheral Blood:

The human peripheral blood products of healthy donors were purchased from Stanford Blood Center. The peripheral blood mononuclear cells (PBMCs) were generated from the residual leukocytes via pheresis (LRS chamber) with the Ficoll-Paque PLUS density gradient media (GE healthcare cat #17144002) according to manufacturer's instruction. The PBMCs were used freshly or were cryopreserved in RPMI media containing 50% FCS serum and 10% DMSO and stored at −80° C. temperately or at −196° C.

### 4. Preparation of Target Cells to Test TCR Antigen Specificity Function:

SKOV3-A2, SKUT1-A2, 293T-A2 and K562-A2 cells were generated by transfection of a recombinant lentiviral vector encoding cDNA for HLA-A*0201 with Lipofectamine 3000 (Thermo Fisher L3000015) according to manufacturer's instructions. The HLA-A*0201 sequence in the lentiviral vector was validated by sequencing. The cells were stained with anti-HLA-A2-PE antibody (Biolegend, clone BB7.2, cat #343305) and were sorted by SH800S cell sorter (Sony Biotechnology). The cDNA encoding the full length mutated p53^{R175H} was generated from KMS26 cells with the SuperScript II Reverse Transcriptase RT-PCR kit (ThermoFisher cat #18064014) and cloned into pCDNA3.3 vector (pCDNA3.3-mp53) according to the manufacturer's suggested protocol. The p53^{R175H} sequence was validated by sequencing. 293T-A2 and K562-A2 cells were transfected with pCDNA3.3-mp53 to transiently express exogenous p53^{R175H}. T2+mt-p53 peptide was the T cells pulsed with the p53^{R175H} peptide (SEQ ID NO:1).

### 5. Peptides:

The mutated p53^{R175H} peptide HMTEVVRHC (SEQ ID NO:1) and wild type p53^{R175} peptide HMTEVVRRC (SEQ ID NO:20) were synthesized by Peptide2.0 (peptide2.com). The peptides were >95% pure as indicated by analytic high-performance liquid chromatography and mass spectrometric analysis. Peptides were dissolved in water at a concentration of 10 mg/mL and stored at −80° C. until use.

6. Induction of p53^{R175H} Peptide Specific CTLs In Vitro:

PBMCs were purified from peripheral blood with Ficoll-Paque Premium (Sigma-Aldrich cat #GE-17-5442-02) according to manufacturer's instructions. A fraction of PBMC preparation was stained with anti-HLA-A2 antibody (Biolegend cat #343303) and analyzed by MACSQuant Analyzer 10 (Miltenyi Biotech). HLA-A2+ PBMCs were used to generate p53^{R175H} peptide specific CTLs. First, dendritic cells were generated according to the protocol in the art. Briefly, the plastic adherent cells from HLA-A2+ PBMCs were cultured in the completed RPMI1640 medium supplemented with 1,000 units/mL recombinant human GM-CSF (Peprotech cat #300-03) and 500 units/mL human IL-4 (Peprotech cat #200-04) for seven days, the immature dendritic cells were then stimulated with human TNF-alpha (ThermoFisher cat #PHC3015) at 10 ng/ml for 48 hours. 105 mature dendritic cells and 2×10⁶ autologous PBMCs per well were co-cultured in 24-well plate with the p53^{R175H} peptide at the final concentration of 5 ug/ml. Human IL-2 at 100u/ml (Peprotech cat #200-02), IL-7 at 5 ng/ml (Peprotech cat #200-07), IL-15 at 5 ng/ml (Peprotech cat #200-15) and IL-21 at 5 ng/ml (Peprotech cat #200-21) were added to the culture after 16-24 hours in 5% CO2 and 37° C. incubation. Half of medium was changed every 3 days with fresh medium containing the cytokine cocktail described above. On the 10-14th day, CD8+ cells were enriched using human CD8+ T Cell Isolation Kit (Miltenyi Biotech cat #130-096-495) and re-stimulated with the peptide antigen. Briefly, 10⁶ purified CD8+ T cells were co-cultured with 2×10⁶ HLA-A2+ autologous PBMCs pre-treated with Mitomycin C (Santa Cruz Biotechnology cat #sc-3514) at 25 μg/ml for 2 hours in 5% CO2 and 37° C. incubation. The p53^{R175H} peptide at 1 ug/ml and the cytokine cocktail described above were added to the culture following the procedure as the 1^{st} antigen stimulation. After 2 cycles of antigen stimulation, the expanded T cells were harvested for analysis and generation of T cell clones.

7. Generation of p53^{R175H} Peptide Specific CTL Clones

To analyze and isolate p53^{R175H}-Specific CTL clones, p53^{R175H}/HLA-A2 tetramer labeled with PE was generated using the p53^{R175H} peptide (SEQ ID No: 1) and Flex-T™ HLA-A*02:01 monomer (Biolegend cat #280003) according to manufacturer's instructions. T cells were harvested and suspended in 50 ul DPBS buffer containing 1 ul of anti-human CD8 antibody labeled with APC (Biolegend cat #300912) and 1 ul of p53^{R175H}/HLA-A2 tetramer labeled with PE. After incubation in 4° C. for 30 minutes, the cells were analyzed by flow cytometry (MACSQuant Analyzer 10) and the data was analyzed by Flowjo software (FlowJo, LLC). T cell clones were generated by single cell sorting with FACS sorter. p53^{R175H} peptide specific CTLs were stained with anti-CD8-APC and p53^{R175H}/HLA-A2 tetramer-PE and resuspended in 400 ul DPBS with 1% FBS, single cell sorting was conducted on Sony SH800 cell sorter according to manufacturer's instructions. The cells were sorted into 96-well U-bottom plates pre-treated overnight in 4° C. with 2 ug/ml anti-CD3 antibody (Biolegend, clone OKT3 cat #317303) and 2 ug/ml anti-CD28 antibody (Biolegend cat #302914). HLA-A2+ PBMCs pre-treated with Mitomycin C (Cayman, cat #11435) at 25 μg/ml for 2 hours were used as feeder cells. The sorted single cells were co-cultured with feeder cells (104 feeder cells per well) in the completed RPMI1640 medium supplemented with IL-2 100u/ml, IL-7, 15 and 21 at 5 ug/ml. Half of medium was changed with fresh medium containing the cytokine cocktail mentioned above every 3 days until the T cells grew to the number enough to be analyzed with CD8 antibody and p53^{R175H}/HLA-A2 tetramer and for isolating TCR gene.

### 8. Cloning TCR Gene:

Total RNA was purified from p53^{R175H} peptide specific CTL clones with Zymo Quick-RNA Microprep kit (Zymo Research cat #R1050) according to manufacturer's instructions. cDNA was generated through RT-PCR with the Smarter RACE 5′/3′ kit (Takara Bio cat #634858). Using the 5′-CDS primer provided by kit and TCR beta chain 3′ primer 5′-GGC AGA CAG GAC CCC TTG CTG G-3′ (SEQ ID NO: 14) or TCR alpha chain 3′ primer 5′-CTT TTC TCG ACC AGC TTG ACA TCA-3′ (SEQ ID NO: 15) to conduct PCR with Q5 High-Fidelity DNA Polymerase (New England Biolabs cat #M0491) according to manufacturer's instructions. The resulted PCR products of TCR alpha chain and beta chain were purified and ligated to pRACE vector provided by Smarter RACE 5′/3′ kit. Plasmids were purified from Stellar Competent Cells (Takara Bio, cat #634858) that were transformed with the ligations of TCR alpha chain and beta chain. Gene sequencing of the inserts on the plasmids was performed to obtain the sequences of TCRV-alpha and V-beta.

### 9. Preparation of the Recombinant TCR Lentiviral Vector:

The inventive TCR gene were cloned into a replication defective lentiviral vector pCDH-EF1α-MCS-PGK-GFP (System Biosciences cat #CD811A-1). The sequences of the V-J region of TCR-alpha and the V-D-J region of TCR-beta of the inventive TCR specific for the p53^{R175H} peptide in the context of HLA-A*02:01 were determined according to the sequences of TCRV-alpha and V-beta generated from each p53^{R175H}-Specific CTL clone. The sequences of mouse TCR-alpha constant chain and mouse TCR-beta constant chain are determined according to the reference sequences (GeneBank KU254562 and EF154514.1 respectively). The nucleic acid with the nucleoid sequence comprising a TCR Vβ chain (SEQ ID No: 36), a nucleic acid encoding mouse TCR-beta constant chain (SEQ ID NO: 27), a TCR Vu chain (SEQ ID NO: 35), a nucleic acid encoding the mouse TCR-alpha constant chain (SEQ ID NO: 28) and a linker nucleic acid encoding a furin enzyme cleavage peptide and a F2A peptide (SEQ ID NO: 13) between the TCR alpha and beta chains was synthesized (Integrated DNA Technologies). The synthesized nucleic acid (SEQ ID NO: 16) was cloned into the multi-cloning site downstream of the EF-1α promoter of the lentiviral vector pCDH-EF1α-MCS-PGK-GFP according to the manufacturer's instructions. The PGK promoter driving the expression of GFP is in the opposite orientation. The nucleotide sequence of the expression cassette is shown in SEQ ID NO: 17. The lentiviral vector expressing a TCR specific for the p53^{R175H} epitope was denoted as pCDH-p53. The inserted nucleic acid was sequenced, and no errors and mutations are found. The lentiviral vector plasmids were transformed into the stellar competent bacteria (Takara Bio cat #636763) to prepare plasmid stocks for making lentivirus particles.

### 10. Preparation of the Recombinant TCR Lentivirus Particles:

TCR lentivirus particles were generated from 293T cells that were transfected with the lentiviral vector pCDH-p53. Briefly, 293T cells growing in 6-well plate were co-transfected with pCDH-p53 plasmid and the pPACKH1-lentivector packaging kit (System Biosciences LV500A-1) by using Lipofectamine 3000 transfection reagent (Invitrogen cat #11668019) according to the manufacturer's instructions. After 48 hours of incubation in 5% CO2 and 37° C., the supernatant was harvested and filtered through a 0.4 μm filter membrane. The virus supernatant was concentrated with Lenti-X™ Concentrator (Takara, cat #631231) according to the manufacturer's instructions. The fresh made TCR-lentivirus was used to infect JRT cells or the activated PBMCs.

### 11. Preparation of the T Cells Expressing the Inventive TCR:

To generate activated human T cells for expressing the inventive TCR, PBMC cells (2×10⁶ per well) were cultured in 24-well plate that was pre-treated with 2 μg/ml of anti-human CD3 antibody (Biolegend cat #317303) and 2 μg/ml of anti-human CD28 antibody (Biolegend cat #302914) in PBS overnight in 4° C. After incubation for 24 hours, the medium was changed with completed RPMI-1640 medium supplemented with the cytokine cocktail IL-2 100 IU/ml, IL-7 5 ng/ml, IL-15 5 ng/ml and IL-21 5 ng/ml. To generate T cell line for expressing the inventive TCR, JRT (J.RT3-T3.5) cell line that is a beta-chain-deficient mutant derived from Jurkat cell line and Jurkat cells that have been transduced to express human CD8 (JurkartCD8) was used. To transfect T cells with the lentivirus encoding the inventive TCR, the activated PBMCs, JRT cells or Jurkat-CD8 cells were resuspended in 24-well plate with 1 ml freshly made lentivirus supernatant or 1 ml completed RPMI 1640 medium containing the concentrated virus. Polybrene (Santa Cruz Biotechnology cat #sc134220) was added at a final concentration of 10 μg/ml. The cells were centrifuged at 1000 g in 32° C. for 2 hours. After incubation in 5% CO2 and 37° C. for 4-6 hours, the medium was changed to completed RPMI-1640 medium supplemented with the cytokine cocktail. Half of medium was replaced every 3 days with fresh medium supplemented with the cytokine cocktail. The cells could also be transfected by using a RetroNectin Dish (RetroNectin Pre-coated Dish, 35 mm) (Takara T110A) according to the manufacturer's instructions. Generally, phenotype and functional analysis could be performed after 72 hours. If the viral vector had GFP, GFP-positive cells could generally be observed under a fluorescence microscope 48-72 hours after transfection. In order to obtain more antigen specific T cells, the transfected T cells could be restimulated with the peptide antigen in the culture containing HLA-A2+ PBMCs that were re-treated with Mitomycin C and pulsed with the p53^{R175H} peptide at 1 ug/ml. After expansion with the cytokine cocktail, the T cells were harvested for further studies.

### 12. Cell Phenotype Analysis by Flow Cytometry:

To analyze the expression of exogenous TCR by PBMCs, JRT cells or Jurkat-CD8 cells transfected with lentiviruses encoding the inventive TCR, the cells are resuspended in DPBS buffer (2.7 mM KCl, 1.5 mM KH2PO4, 136.9 mM NaCl, 8.9 mM Na2HPO4·7H2O, pH 7.4) with 1% FBS and stained with APC labeled anti-human CD8 antibody (Biolegend 300912) and PE labeled p53^{R175H}/HLA-A2 tetramer that was produced as described above. The flow cytometer is a MACSQuant Analyzer 10 (Miltenyi Biotec Corporation), and the results are analyzed by Flowjo software (Flowjo Corporation). To analyze the expression of HLA-A2 by the target cells, the cells were stained with FITC anti-human HLA-A2 Antibody (Biolegend 343303) and analyzed by flow cytometry.

### 13. T Cell Functional Analysis:

To assess the specificity and function of the inventive TCR expressed by JRT cells, the expression of CD69 as an activation marker on JRT cells after antigen stimulation was analyzed by flow cytometry according to the method in the art (Cytometry. 1996; 26(4):305-10). Briefly, in 96-well plate, JRT cells that were transduced with pCDH-p53 lentivirus were co-cultured with the target cells for 16 hours, for example, mixed culture with T2 cells (E:T ratio was 1:1) pulsed with the mutated p53^{R175H} peptide at different concentrations. After incubation, the cells were stained with PE labeled anti-CD69 antibody (Biolegend 310905) and analyzed by flow cytometry. If the TCR expressed by JRT cells could specifically recognize p53^{R175H} peptide in the context of HLA-A2, the JRT cells would be activated and express CD69 on the surface. GFP+ JRT cells represented the cells that were transduced by the lentiviral vector encoding both the inventive TCR and GFP. The frequency of GFP+CD69+ JRT represented the activated T cell population that expressed the inventive TCR. To assess the specificity and function of the inventive TCR that was expressed by the primary T cells in PBMCs, the specific expression of CD137 by the T cells after antigen stimulation was measured by flow cytometry according to the method in the art. Study shows that CD137+ tumor-infiltrating T lymphocytes possessed tumor reactivity in vitro and mediated superior anti-tumor effect in vivo (Clin Cancer Res 2014; 20(1) 44-55). CD137 assay has an excellent correlation with the cytokine flow cytometry assay to assess the secretion of IFNg or TNFa by the activated T cells, as well as with the CD107a-mobilization shift assay to assess degranulation (cytotoxicity) of the activated T cells (Blood 2007; 110(1): 201-210). Briefly, in the 96-well plate, PBMCs that were transduced with the recombinant lentiviral vector (pCDH-p53) were co-cultured with the target cells (E:T ratio was 5:1) for 24 hours, after incubation, the cells were stained with PE labeled anti-CD137 antibody (Biolegend 309803) and APC labeled anti-CD8 antibody and analyzed by flow cytometry. If the TCR expressed by T cells could specifically recognize p53^{R175H} peptide in the context of HLA-A2, the T cells would be activated and express CD137 on the surface. GFP+CD8+ cells represented the cytotoxic T cells that were transduced by the lentiviral vector encoding both the inventive TCR and GFP. The frequency of GFP+CD8+CD137+ T cells represented the CTL population that expressed the inventive TCR and was activated by antigen stimulation.

### Example 1

This example demonstrates that the p53^{R175H} peptide specific cytotoxic T cells (CTLs) can be induced from HLA-A2+ PBMCs that are purified from blood of a healthy donor, and antigen specific CTL clones can be generated from the p53^{R175H} peptide specific CTLs.

Using the method described above, p53^{R175H} peptide specific CTLs were induced from PBMCs after 2 cycles of antigen stimulations with the p53^{R175H} peptide (HMTEVVRHC, SEQ ID NO:1). The resulted T cells were analyzed by flow cytometry.

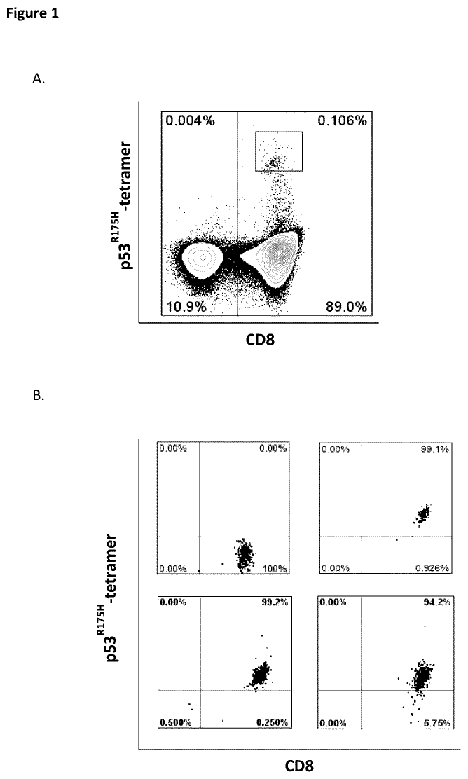

FIG. 1A shows that 0.106% of the living gated lymphocytes were CD8-positive cytotoxic T cells that could bind p53^{R175H}/HLA-A2 tetramer (p53^{R175H}-tetramer). The result shows that in the natural T cell repertoire of a healthy donor, the specific T cells that can recognize the p53^{R175H} peptide in the context of HLA-A2 exist although the frequency of this T cell population is very low. The pre-existence of the p53^{R175H} peptide specific TCRs in the peripheral T cell repertoire of HLA-A2+ normal people suggest the low safety risk of the cross-reaction against normal cells and causing autoimmune reactions by the T cell population with the inventive TCR, which makes T cells with the inventive TCR suitable for adoptive T cell therapy. The p53^{R175H} peptide specific CTLs could be generated in vitro with the p53^{R175H} peptide antigen that was presented by the dendritic cells and expanded with the cytokines including IL-2, IL-7, IL-15 and IL-21. In addition, based on the fluorescence intensity of the p53^{R175H}/HLA-A2 tetramer, the CD8+ tetramer+ T cells contained a diverse T cell population with different TCR binding affinity. A dominant population with high-affinity T cells was induced and expanded. This T cell population was gated and subjected for single cell sorting to generate CTL clones.

Single cells from the gated CD8+ tetramer+ T cells were sorted into each well of a 96-well plate using Sony SH800 FACS sorter. Total 288 CD8+ tetramer+ single T cells were sorted into each well and cultured under the condition described in the above method. After antigen restimulation and expansion with the cytokines, a total of fifty-eight T cell clones grew to reach the number enough for analysis and isolation of TCR gene. A fraction of cells of each T cell clone was harvested and analyzed by flow cytometry. Forty-one clones out of fifty-eight analyzed clones were CD8+p53^{R175H}/HLA-A2 tetramer+, which suggested that these T cell clones were able to recognize the p53^{R175H} peptide in the context of HLA-A2. FIG. 1B shows the flow analysis of four clones that represented total T cell clones that were stained with anti-CD8 antibody and p53^{R175H}/HLA-A2 tetramer. The upper left plot shows a T cell clone that could not bind p53^{R175H}/HLA-A2 tetramer. This T cell clone might be nonspecifically expanded under the culture condition. The other three plots show three T cell clones that could bind p53^{R175H}/HLA-A2 tetramer.

Total RNA was purified from three CD8+p53^{R175H}/HLA-A2 tetramer+ T cell clones. The variable regions of the TCRV-alpha and V-beta were cloned using the method described in the method, and their sequences were identified by gene sequencing. The nucleoid sequence of TCRV-alpha is shown as SEQ ID NO: 35. The nucleoid sequence of TCRV-beta is shown as SEQ ID NO: 36. The TCRs from three different clones turned out to be identical, which demonstrated that T cell clones with this p53^{R175H} peptide specific TCR were predominately expanded from the PBMBs under the culture condition described in the method.

### Example 2

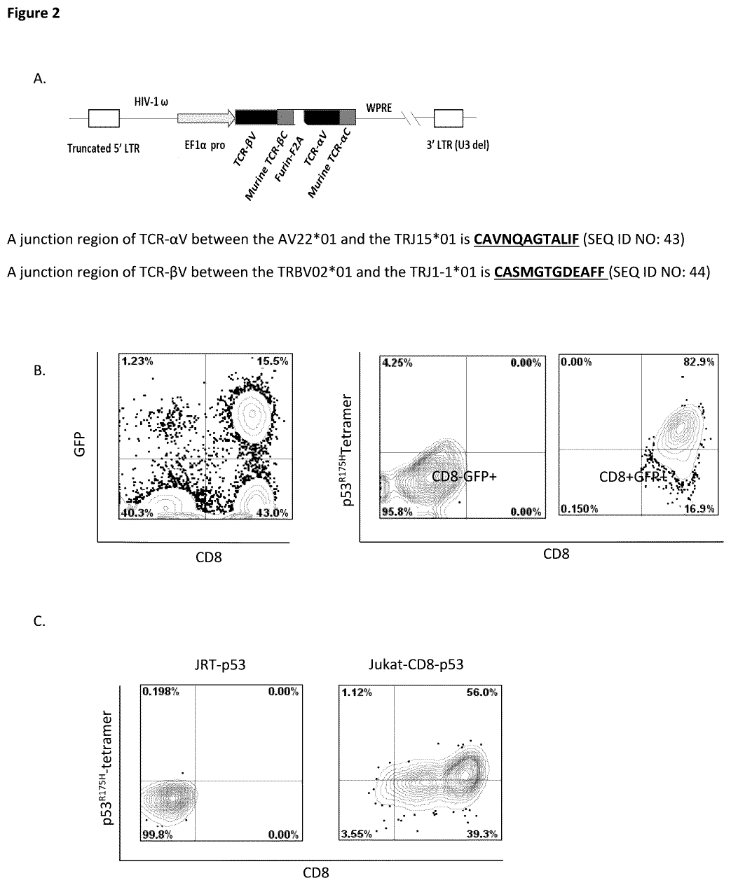

To access the antigen specificity and function of the inventive TCR expressed by T cells, the nucleic acids of α chain and β chain of the inventive TCR were cloned into a replication-deficient lentiviral expression vector. FIG. 2A shows a schematic diagram of the recombinant lentiviral vector comprising encoding the inventive TCR. The constant regions of the human TCR α chain and β chain were replaced with the constant regions of murine α chain and murine β chain respectively. The nucleic acid encoding TCR β chain and α chain were connected by a nucleic acid encoding a cleavable linking polypeptide (SEQ ID NO 13). The expression of the inventive TCR α chain and β chain (SEQ ID NO: 16) was driven by an EF-1α promoter. This promoter is a highly expressed promoter in eukaryotic cells and will not be affected by methylation and other factors that may cause a loss of function and thus is suitable for long-term expression of foreign genes in vivo. TCR α chain and β chain were connected by an F2A sequence so that TCR α chain and β chain genes could be transcribed at the same time and subsequently translated through ribosome skipping to generate a separated TCR α chain polypeptide and a β chain polypeptide. This design ensures equal amounts of the TCR α chain and β chain to enhance the formation of TCR dimers. The furin restriction site between the TCR α and β chains of TCR can remove the excess peptide at the C-terminus of the β chain.

### Example 3

This example demonstrates that primary T cells from healthy donor that express the inventive TCR specific for the mutated p53^{R175H} can recognize the p53^{R175H} peptide in the context of HLA-A2.

In order to access whether the inventive TCR could be expressed in the primary T cells after transfected by the recombinant lentivirus encoding the inventive TCR and GFP (pCDH-p53), PBMC cells from a healthy donor were treated with anti-CD3 and anti-CD28 antibody, and the activated T cells were transfected by the recombinant lentivirus particles carrying the inventive TCR gene using the method described in the method. The cells were harvested after antigen restimulation and expansion with the cytokine cocktail. Anti-CD8 and p53^{R175H}/HLA-A2 tetramer staining was performed. The left graph in FIG. 2B shows that 15.5% of living gated lymphocytes were CD8+GFP+ CTLs, which represented the T cell population that was transduced with the recombinant lentivirus. Since the GFP+ cells were living gated from lymphocyte population, 1.23% of CD8-GFP+ cells were most likely the CD4+ T cell population that were transduced with the recombinant lentivirus.

To access whether the inventive TCR expressed by primary T cells could recognize the mutated p53^{R175H} peptide in the context of HLA-A2, the GFP+ cells were gated and analyzed for the binding of p53^{R175H}/HLA-A2 tetramer. The right graph in FIG. 2B shows the frequency of p53^{R175H}/HLA-A2 tetramer+ cells in the gated CD8-GFP+ population (left plot) and the gated CD8+GFP+ population (right plot). The results show that 82.9% of transduced CD8+ CTLs could bind p53^{R175H}/HLA-A2 tetramer, which demonstrates that the inventive TCR expressed by this CD8+ CTL population could recognize the mutated p53^{R175H} peptide in the context of HLA-A2. However, only a fraction of CD8-GFP+ T cells that represented CD4 T cells could bind p53^{R175H}/HLA-A2 tetramer, which suggests that the binding of p53^{R175H}/HLA-A2 tetramer by the inventive TCR relies on the existence of CD8.

To further access whether the binding of the inventive TCR to p53^{R175H}/HLA-A2 tetramer was CD8-dependent, CD8 negative JRT cells that were derived from the Jurkat line and CD8 positive Jurkat cells (Jurkat-CD8) that were transduced to express exogenous CD8a were transduced to express the inventive TCR and stained with anti-CD8 antibody and p53^{R175H}/HLA-A2 tetramer. GFP+ transduced cells were gated to analyze the binding of p53^{R175H}/HLA-A2 tetramer by the T cells. The left plot of FIG. 2C shows that without CD8 expression, the inventive TCR expressed by JRT cells could not bind the tetramer. However, with the help of CD8, the inventive TCR expressed by Jurkat-CD8 cells could bind the tetramer (FIG. 2C right plot). This result demonstrates that the inventive TCR requires CD8 on T cells as the co-receptor to bind the soluble p53^{R175H}/HLA-A2 tetramer. Although the precise affinity threshold of TCR that makes a CD8-independent HLA/peptide tetramer binding is not yet defined, studies show that the minimal K_{D} threshold of TCR affinity for CD8-independence approximately ranges from 3 to 6 μM (Immunology. 2009; 126(2): 165-176), which suggests that the inventive TCR might have a binding affinity to the p53^{R175H} peptide in the context of HLA-A2 not higher than the TCR with K_{D} value of 3 to 6 μM.

### Example 4

The functional analysis of the inventive TCR in this example demonstrates that the inventive TCR expressed by T cells can recognize the p53^{R175H} peptide in the context of HLA-A2 with high avidity.

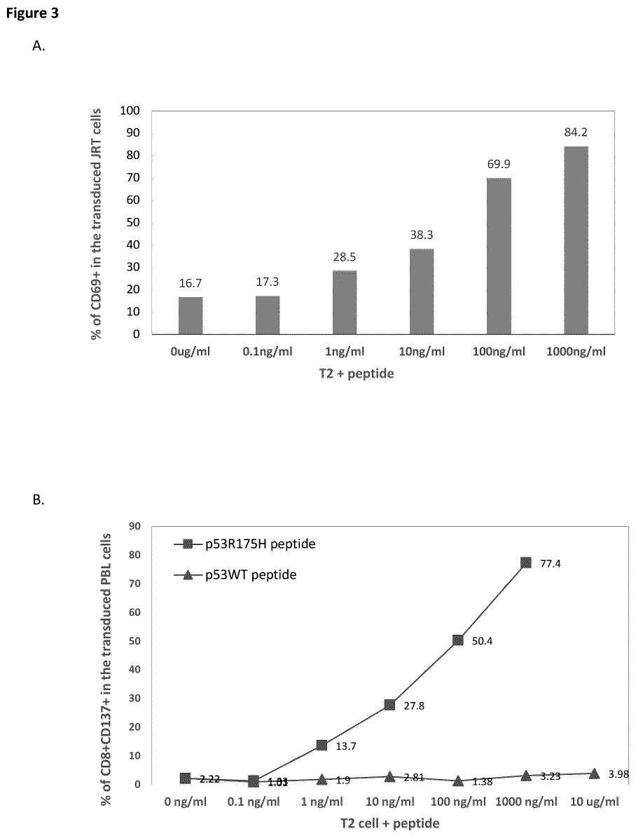

To assess the function of the inventive TCR expressed by JRT cells, JRT cells were transduced with the recombinant lentivirus encoding the inventive TCR (pCDH-p53) and co-cultured with T2 cells pulsed with 10× dilution series of the p53^{R175H} peptide starting from 1 ug/ml. After antigen stimulation for 16 hours, the GFP+ cells were gated and the percentages of CD69+ JRT cells were analyzed by flow cytometry. FIG. 3A shows that the JRT cells transduced with pCDH-p53 expressed CD69 after stimulation with the antigenic peptide, which demonstrated that JRT cells were activated by the p53^{R175H} peptide presented by HLA-A2 and the activation was a peptide dose-dependent response. JRT cells could be activated by p53^{R175H} peptide at as low as the concentration of 10 ng/ml based on the twice fold increase of the frequency of CD69+ cells compared to the control group without adding p53^{R175H} peptide. The result demonstrates that without the co-receptor CD8, the inventive TCR on T cells can effectively recognize the p53^{R175H} peptide at low concentration. The avidity of the inventive TCR against its cognate antigenic peptide is comparable to the high-avidity TCRs specific to neo-epitopes reported in other studies (Cancer Immunol Res. 2019; 7(4): 534-543).

To assess the function of the inventive TCR expressed by primary T cells, the activated PBMC cells were transduced with the recombinant lentivirus encoding the inventive TCR (pCDH-p53) and co-cultured with T2 cells pulsed with 10× dilution series of the mutated p53^{R175H} peptide starting from 1 ug/ml, or with 10× dilution series of the wild type p53^{R175} peptide starting from 10 ug/ml. After antigen stimulation for 24 hours, the GFP+ cells were gated and the percentages of CD8+CD137+ T cells were analyzed by flow cytometry. FIG. 3B shows that the TCR-gene transduced CTLs could be activated by the stimulation with the mutated p53^{R175H} peptide presented by HLA-A2. The activation was a peptide dose-dependent response. The transduced T cells could be activated by the p53^{R175H} peptide at as low as the concentration of 1 ng/ml based on the twice fold increase of the frequency of CD8+CD137+ cells compared to the control group without adding the p53^{R175H} peptide. The inventive TCR expressed by the transduced T cells could not recognize the wild type p53^{R175} peptide even at high concertation of 10 μg/ml. This result demonstrates that the inventive TCR can specifically recognize the mutated p53^{R175H} epitope peptide presented by HLA-A2 while ignoring the wild type counterpart epitope in the context of HLA-A2 at high concentration. Co-receptor CD8 on the T cells enhance the avidity of the inventive TCR by 10-fold increase compared to the TCR on CD8-negative JRT cells.

There was a study showing that TCR functional potency are not determined by TCR affinity alone, but by avidity determined by the combined contribution of TCR and CD8. TCR affinity contributes to avidity up to the 10-μM threshold after which further increase does not lead to higher avidity and consequent stronger T-cell functions (PNAS 2013; 110 (17): 6973-6978). The inventive TCR may not have super-high affinity that allow it to bind p53^{R175H}/HLA-A2 tetramer without CD8 help, however, it was still able to recognize the p53^{R175H} peptide with high avidity compared to the other reported high avidity TCRs. In addition, the proper affinity of the inventive TCR may avoid the safety concerns related to the potential cross-reactive autoimmunity caused by TCRs with super high affinity. The high avidity of the inventive TCR demonstrated in the examples allows it to detect very low expression of the p53^{R175H} epitope peptide presented by HLA-2 on target cells such as cancer cells with the mutated p53^{R175H}.

### Example 5

This example demonstrates that that HLA-A2+ tumor cells with the mutated p53^{R175H} can be recognized by the primary T cells that are transduced to express the inventive TCR.

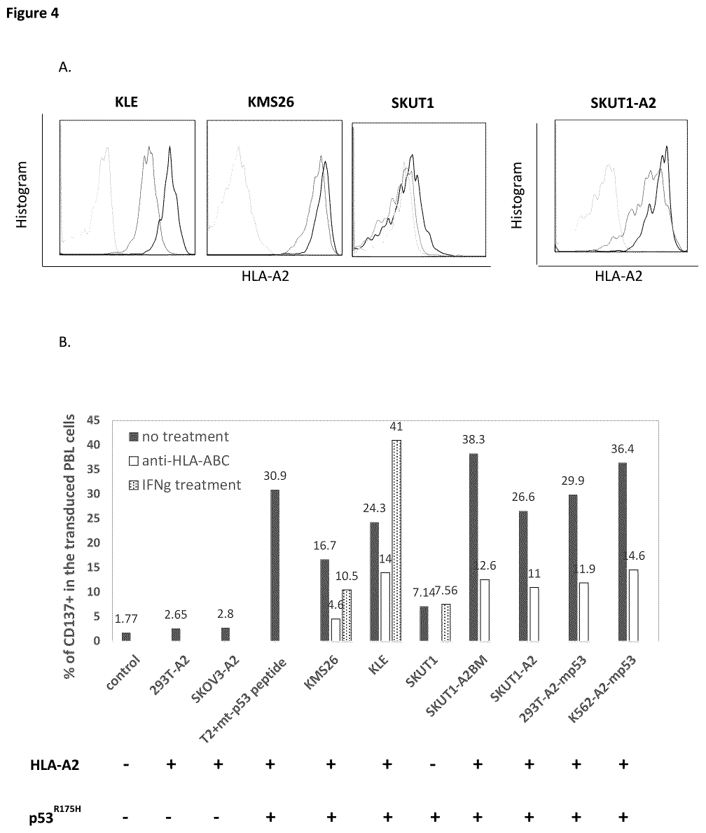

The expression of HLA-A2 on the tumor cell lines was accessed with PE labeled anti-HLA-A2 antibody (Biolegend cat #343305). Tumor cell lines KLE, KMS26 and SKUT1 were pre-treated with human IFN-gamma (200u/ml) for 24 hours in 5% CO2 and 37 C incubation. The tumor cells with or without pre-treatment of IFN-gamma were stained with anti-HLA-A2 antibody and analyzed by flow cytometry. The left graph in FIG. 4A shows the expression of HLA-A2 by the tumor cell lines, including the control group that was stained with PE labeled isotype antibody (light gray line), tumor cells that were not treated with IFN-gamma (medium gray line) and tumor cells that were pre-treated with IFN-gamma (dark gray line). The result demonstrates that the tumor cell lines KLE and KMS26 express HLA-A2 on the cell surface and the expression of HLA-A2 can be enhanced by IFN-gamma stimulation, particularly for KLE cells. SKUT1 cell line does not express HLA-A2 on the surface although this cell line has a heterozygous HLA-A2 allele according to TRON Cell Line Portal. IFN-gamma treatment cannot upregulate HLA-A2 expression on SKUT1 cells. To generate HLA-A2+ SKUT1 cells, SKUT1 cells were transduced with a lentiviral vector encoding HLA-A2 or HLA-A2/beta 2-microglubine. The right plot shows that SKUT1 cells transduced with HLA-A2 alone (medium gray line) or HLA-A2 together with beta 2-microglubine (dark gray line) can express HLA-A2 on the surface. Besides SKUT1 cell line, SKOV3, 293T and K562 cell lines were also transduced to express exogenous HLA-A2.

According to TRON Cell Line Portal, KLE, KMS26 and SKUT1 cell line carry a mutated p53 p.R175H. The transcript expression of p53 in these cell lines provided as ‘Reads Per Kilobase of transcript, per Million mapped reads’ (RPKM) is reported as the following: KMS26 line expresses p53 at 187.2 RPKM, KLE line expresses p53 at 232.4 RPKM and SKUT1 expresses p53 at 310.2 RPKM.

To assess the function of the inventive TCR expressed by primary T cells to recognize tumor cells, the activated PBMC cells were transduced with the recombinant lentivirus encoding the inventive TCR (pCDH-p53) and co-cultured with a variety of target cell lines including tumor cells expressing HLA-A2 alone, the mutated p53^{R175H} alone, or both HLA-A2 and the mutated p53^{R175H}. T2 cells pulsed with the p53^{R175H} peptide at 100 ng/ml was the positive control. The transduced PBMCs without target cells was the negative control. After antigen stimulation for 24 hours, the GFP+ cells were gated and the percentages of CD8+CD137+ T cells were analyzed by flow cytometry. FIG. 4B shows that the inventive TCR expressed by the primary CTL cells could be activated by recognizing the HLA-A2+p53^{R175H}+target cells (filled bars), including KMS26, KLE, SKUT1 that was transduced to express exogenous HLA-A2, 293T-A2 that was transduced to express mutated p53^{R175H} and K562-A2 that was transduced to express mutated p53^{R175H}. Target cells that express HLA-A2 alone (293T-A2 and SKOV3-A2) could not activate the T cells with the inventive TCR. HLA-A2 negative SKUT1 could not activate the T cells with the inventive TCR even it was stimulated with IFN-gamma, however, SKUT1-A2 expressing exogenous HLA-A2 could activate the T cells with the inventive TCR, which demonstrates that the endogenous p53^{R175H} epitope peptide can be presented by the exogenous HLA-A2 on the SKUT1-A2 cells and be recognized by the inventive TCR. Anti-HLA-ABC antibody (Biolegend cat #311427) were added into the mixed culture and shew that blocking HLA-I could inhibit the expression of CD137 on the CTLs with the inventive TCR (open bars), which further illustrated that the inventive TCR was activated by the antigenic peptide presented by HLA class I molecule. Pre-treatment with IFN-gamma could enhance HLA-A2 expression on KLE cells, which may explain the result that IFN-gamma pre-treated KLE cells activated the inventive TCR at higher level. However, IFN-gamma pre-treated KMS26 cells activated T cells with the inventive TCR at lower level. The reason remains unknown but the possible upregulated PD-L1 on KMS26 after IFN-gamma stimulation might be speculated. This example demonstrates that the inventive TCR expressed by primary T cells has a sensitivity high enough to detect the low expression of endogenous p53^{R175H} epitope peptide presented by HLA-A2 on tumor cells.

### Example 6

This example demonstrates the amino acid sequences of the TCR of this invention, including the TCR constructed in Example 2. The amino acid sequences of the TCR alpha and beta chain of these TCRs are shown in Table 1. The CDRs are bold and underlined.

TABLE 1

| TCR NAME | AMINO ACID SEQUENCE (SEQ ID NO) |
|---|---|
| Alpha chain variable | MKRILGALLGLLSAQVCCVRGIQVEQSPPDLILQE- |
| region (with | GANSTLRCNFSDSVNNLQWFHQNPWGQLINLFYIPSGTKQNGRLSATTVATERYSLLYISSSQTT |
| N-terminal signal | DSGVYFCAVNQAGTALIFGK GTTLSVSSN |
| peptide) | (SEQ ID NO: 11) |
| Alpha chain variable | GIQVEQSPPDLILQEGANSTLRCNFSDSVNNLQWFHQNPWGQLINLFYIPSGTKQNGRL- |
| region (without | SATTVATERYSLLYISSSQTTDSGVYFCAVNQAGTALIFGK GTTLSVSSN (SEQ ID NO: 24) |
| N-terminal | |
| signal peptide) | |
| Beta chain variable | MDTWLVCWAIFSLLKAGLTEPEVTQTPSHQVTQMGQEVIL- |
| region (with | RCVPISNHLYFYWYRQILGQKVEFLVSFYNNEISEKSEIFDDQFSVERPDGSNFTLKIRSTKLEDS |
| N-terminal signal | AMYFCASMGTGDEA FFGQGTRLTVV (SEQ ID NO: 12) |
| peptide) | |
| Beta chain variable | EPEVTQTPSHQVTQMGQEVIL- |
| region (without | RCVPISNHLYFYWYRQILGQKVEFLVSFYNNEISEKSEIFDDQFSVERPDGSNFTLKIRSTKLEDS |
| N-terminal | AMYFCASMGTGDEAFFGQGTRLTVV (SEQ ID NO: 23) |
| signal peptide) | |
| Alpha chain variable | MKRILGALLGLLSAQVCCVRGIQVEQSPPDLILQE- |
| region with | GANSTLRCNFSDSVNNLQWFHQNPWGQLINLFYIPSGTKQNGRLSATTVATERYSLLYISSSQTT |
| human alpha | DSGVYFCAVNQAGTALIFGKGTTLSVSSNIQNPDPAVYQLRDSKSSDKSVCLFTDFD- |
| chain constant | SQTNVSQSKDSDVYITDKTVLDMRSMDFKSNSAVAWSNKSDFACANAFNNSIIPEDTFFPSPESS |
| region | CDVKLVEKSFETDTNLNFQNLSVIGFRILLLKVAGFNLLMTLRLWSS (SEQ ID NO: 47) |
| Alpha chain variable | MKRILGALLGLLSAQVCCVRGIQVEQSPPDLILQE- |
| region with | GANSTLRCNFSDSVNNLQWFHQNPWGQLINLFYIPSGTKQNGRLSATTVATERYSLLYISSSQTT |
| murine alpha | DSGVYFCAVNQAGTALIFGKGTTLSVSSNIQNPEPAVYQLKDPRSQDSTLCLFTDFD- |
| chain constant | SQINVPKTMESGTFITDKTVLDMKAMDSKSNGAIAWSNQTSFTCQDIFKETNATYPSSDVPCDAT |
| region | LTEKSFETDMNLNFQNLSVMGLRILLLKVAGFNLLMTLRLWSS (SEQ ID NO: 5) |
| Alpha chain variable | MKRILGALLGLLSAQVCCVRGIQVEQSPPDLILQE- |
| region with | GANSTLRCNFSDSVNNLQWFHQNPWGQLINLFYIPSGTKQNGRLSATTVATERYSLLYISSSQTT |
| human delta | DSGVYFCAVNQAGTALIFGKGTTLSVSSNS- |
| chain constant | QPHTKPSVFVMKNGTNVACLVKEFYPKDIRINLVSSKKITEFDPAIVISPSGKYNAVKLGKYEDSNS |
| region | VTCSVQHDNKTVHSTDFEVKTDSTDHVKPKETENTKQPSKSCHKPKAIVHTEKVNMM- |
| | SLTVLGLRMLFAKTVAVNFLLTAKLFFL (SEQ ID NO: 49) |
| Beta chain variable | MDTWLVCWAIFSLLKAGLTEPEVTQTPSHQVTQMGQEVIL- |
| region with | RCVPISNHLYFYWYRQILGQKVEFLVSFYNNEISEKSEIFDDQFSVERPDGSNFTLKIRSTKLEDS |
| human beta | AMYFCASMGTGDEAFFGQGTRLTVVDLNKVFPPEVAVFEPSEAEISHTQKATLVCLATGFF- |
| chain constant | PDHVELSWWVNGKEVHSGVSTDPQPLKEQPALNDSRYCLSSRLRVSATFWQNPRNHFRCQVQ |
| region | FYGLSENDEWTQDRAKPVTQIVSAEAWGRADCGFTSVSYQQGVLSATILYEILLGKAT- |
| | LYAVLVSALVLMAMVKRKDF (SEQ ID NO: 48) |
| Beta chain variable | MDTWLVCWAIFSLLKAGLTEPEVTQTPSHQVTQMGQEVIL- |
| region with | RCVPISNHLYFYWYRQILGQKVEFLVSFYNNEISEKSEIFDDQFSVERPDGSNFTLKIRSTKLEDS |
| murine beta | AMYFCASMGTGDEAFFGQGTRLTVVEDLRNVTPPKVSLFEPSKAEIANKQKATLVCLARGFF- |
| chain constant | PDHVELSWWVNGKEVHSGVSTDPQAYKESNYSYCLSSRLRVSATFWHNPRNHFRCQVQFHGL |
| region | SEEDKWPEGSPKPVTQNISAEAWGRADCGITSASYQQGVLSATILYEILLGKAT- |
| | LYAVLVSTLVVMAMVKRKNS (SEQ ID NO: 6) |

### CONCLUSION

In the present invention, a novel TCR that is specific for the mutated p53^{R175H} epitope peptide (SEQ ID NO:1) in the context of HLA-A2 has been identified and generated from a healthy human donor. The inventive TCR has a high avidity to recognize the exogenous as well as endogenous p53^{R175H} epitope peptide presented by HLA-A2 on target cells but has no response to the counterpart wild type p53^{R175} peptide. The antigen specificity and high avidity against p53^{R175H} makes the inventive TCR suitable for the development of immunotherapy regiments specifically targeting cancer that carries HLA-A2 allele and the mutated p53^{R175H}. T cells that are transduced to express the inventive TCR exogenously can be applied for adoptive T cell therapy to treat patients with cancer.

1. An isolated or purified T-cell receptor (TCR) comprising:
an α chain and a β chain, each one of the α and β chains comprising a first, second and third complementarity determining regions (CDRs), wherein the first CDR (CDR1) of the α chain comprises an amino acid sequence: DSVNN (SEQ ID NO: 7), wherein the second CDR (CDR2) of the α chain comprises an amino acid sequence: IPSGT (SEQ ID NO: 8), wherein the third CDR (CDR3) of the α chain comprises an amino acid sequence: CAVNQAGTALI (SEQ ID NO: 3), wherein the CDR1 of the β chain comprises an amino acid sequence: SNHLY (SEQ ID NO: 9), wherein the CDR2 of the β chain comprises an amino acid sequence: FYNNEI (SEQ ID NO: 10), and wherein the CDR3 of the β chain comprises an amino acid sequence: CASMGTGDEAF (SEQ ID NO: 4).

2. The isolated or purified TCR of claim 1, wherein the α chain further comprises an α chain variable region comprising an amino acid sequence of SEQ ID NO: 11 or SEQ ID NO: 24, and wherein the β chain further comprises an amino acid sequence of a β chain variable region comprising an amino acid sequence of SEQ ID NO: 12 or SEQ ID NO: 23.

3. The isolated or purified TCR of claim 1, further comprising an α chain constant region comprising an amino acid sequence of SEQ ID NO: 26, SEQ ID NO: 28 or SEQ ID NO:45, and a β chain constant region comprising an amino acid sequence of SEQ ID NO: 25, SEQ ID NO: 27 or SEQ ID NO:46.

4. The isolated or purified TCR of claim 1, further comprising (a) the α chain comprising an amino acid sequence of SEQ ID NO: 5 and the β chain comprising an amino acid sequence of SEQ ID NO: 6, (b) the α chain comprising an amino acid sequence of SEQ ID NO: 47 and the β chain comprising an amino acid sequence of SEQ ID NO: 48, or (c) the α chain comprising an amino acid sequence of SEQ ID NO: 49 and the β chain comprising an amino acid sequence of SEQ ID NO: 50.

5. The isolated or purified TCR of claim 1 further comprising an antigenic specificity for a mutated p53^{R175H} peptide HMTEVVRHC (SEQ ID NO: 1) in a context of an HLA-A*02 molecule.

6. The isolated or purified TCR of claim 1, wherein an isolated or purified polypeptide comprises a functional portion of the TCR, and wherein the functional portion comprises amino acid sequences of (a) SEQ ID NO: 11, (b) SEQ ID NO: 12, (c) SEQ ID NO: 23, (d) SEQ ID NO: 24, (e) both SEQ ID NOs: 11 and 12, or (f) both SEQ ID NOs: 23 and 24.

7. The isolated or purified TCR of claim 6, wherein the functional portion comprises amino acid sequences of (a) SEQ ID NO: 5, (b) SEQ ID NO: 6, (c) SEQ ID NO: 47, (d) SEQ ID NO: 48, (e) SEQ ID NO: 49, (f) SEQ ID NO: 50, (g) both SEQ ID NOs: 5 and 6, (h) both SEQ ID NOs: 47 and 48, or (i) both SEQ ID NOs: 49 and 50.

8. The isolated or purified TCR of claim 1, wherein the TCR is included in an isolated or purified protein, the isolated or purified protein comprising a first polypeptide chain comprising amino acid sequences of SEQ ID NO: 3, 7 and 8, and a second polypeptide chain comprising amino acid sequences of SEQ ID NO: 4, 9 and 10.

9. The isolated or purified TCR of claim 8, wherein the isolated or purified protein comprises (a) a first polypeptide chain comprising an amino acid sequence of SEQ ID NO: 11 and a second polypeptide chain comprising an amino acid sequence of SEQ ID NO: 12, or (b) an amino acid sequence of SEQ ID NO: 23 and a second polypeptide chain comprising an amino acid sequence of SEQ ID NO: 24.

10. The isolated or purified TCR of claim 8, wherein the isolated or purified protein comprises (a) a first polypeptide chain comprising an amino acid sequence of SEQ ID NO: 5 and a second polypeptide chain comprising an amino acid sequence of SEQ ID NO: 6, (b) an amino acid sequence of SEQ ID NO: 47 and a second polypeptide chain comprising an amino acid sequence of SEQ ID NO: 48, or (c) an amino acid sequence of SEQ ID NO: 49 and a second polypeptide chain comprising an amino acid sequence of SEQ ID NO: 50.

11. The isolated or purified TCR of claim 1, wherein the TCR is included in an isolated or purified nucleic acid, the isolated or purified nucleic acid comprising a nucleotide sequence that encodes the TCR.

12. The isolated or purified TCR of claim 11, wherein the isolated or purified nucleic acid comprises a nucleotide sequence selected from the group consisting of: SEQ ID NO: 16, 17, 35, 36, 37, 38, 39, 40, 41 and 42.

13. The isolated or purified TCR of claim 12, wherein the isolated or purified nucleic acid is included in a recombinant expression vector.

14. The isolated or purified TCR of claim 13, wherein the recombinant expression vector comprises the isolated or purified nucleic acid.

15. The isolated or purified TCR of claim 13, wherein the recombinant expression vector is selected from the group consisting of: a mRNA, a circular RNA, a single-stranded DNA, a double-stranded DNA, a plasmid, a transposon or a viral vector.

16. The isolated or purified TCR of claim 15, wherein an isolated or purified host cell comprises the nucleic acid or the recombinant expression vector, wherein the host cell is a human lymphocyte, wherein the lymphocyte is selected from the group consisting of a T cell, a natural killer T (NKT) cell, an invariant natural killer T (iNKT) cell, and a natural killer (NK) cell.

17. The isolated or purified TCR of claim 16, wherein the host cell is autologous to a mammal that is treated with the host cell or is allogeneic to the mammal that is treated with the host cell.

18. The isolated or purified TCR of claim 16, wherein the isolated or purified host cell is used to prepare a pharmaceutical composition.

19. The isolated or purified TCR of claim 18, wherein the TCR is included in the pharmaceutical composition that can be used for treating or preventing cancer in a mammal, and wherein the cancer is known to comprise an R175H mutation in human p53 and an HLA-A2 allele.

20. The isolated or purified TCR of claim 1 further comprising an antigenic specificity for a mutated p53^{R175H} peptide HMTEVVRHC (SEQ ID NO: 1) in a context of an HLA-A*02 molecule.
